(19) United States
(12) Patent Application Publication
(10) Pub. No.: US 2021/0077617 A1
(43) Pub. Date: Mar. 18, 2021

(54) IMMUNE MODULATION

(71) Applicant: N-Fold LLC, Fairfield, CT (US)

(72) Inventors: Howard B. Sosin, Fairfield, CT (US); Michael J. Caplan, Woodbridge, CT (US)

(21) Appl. No.: 16/960,869

(22) PCT Filed: Jan. 8, 2019
(86) PCT No.: PCT/US19/12634

§ 371 Date: Jul. 8, 2020

Related U.S. Application Data
(60) Provisional application No. 62/615,088, filed on Jan. 9, 2018.

(51) Int. Cl. A61K 39/35; A61K 39/39

(52) U.S. Cl. CPC A61K 39/35; A61K 2039/57; A61K 39/39

(57) ABSTRACT

The disclosure relates to compositions, methods of use and making, and kits for immunomodulation. In some embodiments, the present invention also provides compositions including at least one antigen substantially co-localized with at least one adjuvant agent in a format selected from patch, capsule, tablet, gel, matrix, paste, reservoir, adhesive, liquid, suspension, lyophilized solid, liquid contained within one or more lumenal units, and combinations thereof, wherein one or both of the antigen and the adjuvant agent is a crude preparation, and wherein the composition is characterized in that administration to a subject in need thereof results in an immune response in the subject to the antigen.

### BACKGROUND

While the immune system is powerful and remarkably adaptive, at times immune reactions can have detrimental effects on the body. For example, anaphylactic responses can result in significant harmful effects, ranging from rash and/or shortness of breath to death. Also, autoimmune reactions, in which an organism's immune system reacts to (e.g., by attacking or otherwise causing damage or detriment) to itself (e.g., to antigens, cells, tissues, etc. that are naturally expressed by the organism) and can result in conditions ranging from acute and/or chronic illness to death.

### SUMMARY

The present disclosure embraces and extends an insight that crude preparations of antigens and/or of adjuvant agents (e.g. via co-localization of antigens and/or adjuvants) can provide valuable immunomodulation including for treatment, e.g. of certain diseases, disorders or conditions.

Various strategies currently employed for immunomodulation involve administration of an antigen and/or of an adjuvant agent. Typically, such antigen agents are administered as substantially pure entities (i.e., separated from other components with which they may occur in nature and/or with which they are associated when produced). The present disclosure encompasses the surprising recognition that use of isolated and/or pure antigens and/or adjuvant agents may limit and/or alter immunomodulatory effectiveness, and that use of more crude materials (e.g., that include the relevant antigen or adjuvant agent in combination with one or more materials with which it is associated in nature or when it is produced, optionally in absolute or relative amounts comparable to that in which they are present in a relevant source), may in fact provide more effective immunomodulation in some circumstances.

Among other things, the present disclosure appreciates that certain insights relevant to features of crude antigen and/or adjuvant agent preparations as described in International Patent Application WO2014/165679 to Allertein Therapeutics, LLC, for Novel Nanoparticle Compositions in the context of nanoparticle compositions, may also prove relevant in other contexts, and particularly in other contexts where antigen and adjuvant agent are maintained in physical proximity (e.g., in the context of a device or composition that co-localizes the antigen and adjuvant agent for administration to a subject).

In some embodiments, the present invention provides compositions including at least one antigen substantially co-localized with at least one adjuvant agent in a format selected from patch, capsule, tablet, gel, matrix (e.g., a chewing gum), paste, reservoir, adhesive, liquid, suspension, lyophilized solid, liquid contained within one or more lumenal units, and combinations thereof, wherein one or both of the antigen and the adjuvant agent is a crude preparation, and wherein the composition is characterized in that administration to a subject in need thereof achieves a desired immunological effect in the subject with respect to the antigen.

In some embodiments, the present disclosure provides compositions including at least one Th2-associated antigen, further associated with at least one crude adjuvant agent, wherein the composition is characterized in that administration to a subject in need thereof results in an immune response in the subject that is not predominantly Th2. In some embodiments, the present disclosure also provides methods including the step of associating at least one Th2-associated antigen combined with at least one crude adjuvant agent.

In some embodiments, the present disclosure provides compositions including at least one Th1-associated antigen, further associated with at least one crude adjuvant agent, wherein the composition is characterized in that administration to a subject in need thereof results in an immune response in the subject that is not predominantly Th1. In some embodiments, the present disclosure also provides methods including the step of associating at least one Th1-associated antigen combined with at least one crude adjuvant agent.

In some embodiments, an antigen is or comprises an allergic antigen. In some embodiments, an antigen is or comprises an anaphylactic antigen. In some embodiments, an antigen is or comprises an infectious antigen. In some embodiments, an antigen is or comprises an autoantigen. In some embodiments, an antigen is or comprises a disease-associated antigen.

The present disclosure further recognizes that in some embodiments a format that facilitates transdermal administration may be useful. In some embodiments, such a format may be or include a patch, gel, paste, adhesive, and combinations thereof.

In some embodiments, an antigen is a Th2-associated antigen. In some such embodiments, an immune response is not a predominantly Th2 response. In some embodiments, an antigen is a Th1-associated antigen. In some such embodiments, an immune response is not a predominantly Th1 response.

In some embodiments, an immune response may be or comprise a predominantly Th1 response. In some embodiments, an immune response may be or comprise a predominantly Th2 response. In some embodiments, an immune response may be or comprise a Treg response. In some embodiments, an immune response may be or comprise at Th17 response.

In some embodiments, provided compositions may be designed and constructed to deliver both an antigen and an adjuvant to a population of a subject's cells. In some embodiments, a population of the subject's cells is or includes immune cells. In some embodiments, immune cells are or include antigen presenting cells (“APCs”).

In some embodiments, provided are methods that include a step of administering a composition provided herein to a subject in need thereof. In some embodiments, a subject is suffering from a disease, disorder or condition associated with an undesirable immune response.

In some embodiments, the present disclosure also provides kits including at least one antigen and at least one adjuvant agent, wherein one or both of the at least one antigen and at least one adjuvant agent is provided as a crude preparation. In some embodiments, antigen and/or adjuvant agent are provided in separate compartments or containers. In some embodiments, a kit further comprises one or more components of an article (e.g., a device or composition) in which antigen and adjuvant may be co-localized for administration as described herein.

Other features, objects, and advantages of the present disclosure are apparent in the detailed description that follows. It should be understood, however, that the detailed description, while indicating embodiments of the present disclosure, is given by way of illustration only, not limitation. Various changes and modifications within the scope of the disclosure will become apparent to those skilled in the art from the detailed description.

### BRIEF DESCRIPTION OF THE DRAWING

FIG. 1 shows a table of exemplary antigens.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included. As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

Allergen: The term “allergen”, as used herein, refers to those antigens that induce an allergic reaction. In some embodiments, an allergen is or comprises a polypeptide. In some embodiments, an allergen is or comprises a small molecule. In some embodiments, an allergen is selected from the group consisting of food allergens, drug allergens, environmental allergens, insect venoms, animal allergens, and latex. Allergic reaction: The phrase “allergic reaction,” as used herein, has its art-understood meaning and refers to an IgE-mediated immune response to an antigen. When an antigen induces IgE antibodies, they will bind to IgE receptors on the surface of basophils and mast cells. Subsequent exposures to the antigen trigger cross-linking of such surface-bound anti-allergen IgEs, which trigger release of histamine from stores within the cells. This histamine release triggers the allergic reaction. Typically, an allergic reaction involves one or more of the cutaneous (e.g., urticaria, angiodema, pruritus), respiratory (e.g., wheezing, coughing, laryngeal edema, rhinorrhea, watery/itching eyes), gastrointestinal (e.g., vomiting, abdominal pain, diarrhea), and/or cardiovascular (e.g., if a systemic reaction occurs) systems. For the purposes of the present disclosure, an asthmatic reaction is considered to be a form of allergic reaction. In some embodiments, allergic reactions are mild; typical symptoms of a mild reaction include, for example, hives (especially over the neck and face) itching, nasal congestion, rashes, watery eyes, red eyes, and combinations thereof. In some embodiments, allergic reactions are severe and/or life threatening; in some embodiments, symptoms of severe allergic reactions (e.g., anaphylactic reactions) are selected from the group consisting of abdominal pain, abdominal breathing sounds (typically high-pitched), anxiety. chest discomfort or tightness, cough, diarrhea, difficulty breathing, difficulty swallowing, dizziness or light-headedness, flushing or redness of the face, nausea or vomiting, palpitations, swelling of the face, eyes or tongue, unconsciousness, wheezing, and combinations thereof. In some embodiments, allergic reactions are anaphylactic reactions.

Allergy: The term “allergy”, as used herein, refers to a condition characterized by an IgE-mediated immune response to particular antigens. In some embodiments, the antigens are ones that do not elicit an IgE-mediated immune response in many or most individuals. In some embodiments, the term “allergy” is used to refer to those situations where an individual has a more dramatic IgE-mediated immune response when exposed to a particular antigen than is typically observed by members of the individual's species when comparably exposed to the same antigen. Thus, an individual who is suffering from or susceptible to “allergy” is one who experiences or is at risk of experiencing an allergic reaction when exposed to one or more allergens. In some embodiments, symptoms of allergy include, for example, presence of IgE antibodies, reactive with the allergen(s) to which the individual is allergic, optionally above a particular threshold, in blood or serum of the individual. In some embodiments, symptoms of allergy include development of a wheal/flare larger than a control wheal/flare when a preparation of the antigen is injected subcutaneously under the individual's skin. In some embodiments, an individual can be considered susceptible to allergy without having suffered an allergic reaction to the particular allergen in question. For example, if the individual has suffered an allergic reaction, and particularly if the individual has suffered an anaphylactic reaction, to a related allergen (e.g., one from the same source or one for which shared allergies are common), that individual may be considered susceptible to allergy to (and/or to an allergic or anaphylactic reaction to) the relevant allergen. Similarly, if members of an individual's family react to a particular allergen, the individual may be considered to be susceptible to allergy to (and/or to an allergic and/or anaphylactic reaction to) that allergen.

Alloantigen: The term “alloantigen”, as used herein, refers to an antigen associated with allorecognition and/or graft rejection (e.g., an antigen against which a rejection immune response is directed). In general, alloantigens are agents that are present in or on tissue from one individual (e.g., a donor individual) of a particular species, but not in or on tissue from another individual (e.g., a recipient individual, for example who is genetically different from the donor individual) of the species, so that transfer of tissue from the donor individual to the recipient individual risks and/or results in a rejection immune response. In general, an antigen may be or include any chemical entity such as, for example, a small molecule, a nucleic acid, a polypeptide, a carbohydrate, a lipid, etc. In some embodiments, an alloantigen is or comprises a polypeptide. A variety of polypeptides are known in the art whose amino acid sequences can vary between and among individuals of the same species such that they might act as alloantigens.

Allorecognition: The term “allorecognition”, as used herein, typically refers to an immune response mounted by the immune system of an individual (i.e., a recipient) who receives a tissue graft from another individual (i.e., a donor, who for example is genetically distinct from the recipient individual) of the same species, which immune response involves recognition of an alloantigen on the grafted tissue. Typically, allorecognition involves T cell recognition of the alloantigen. In many embodiments, T cells recognize an alloantigen peptide, for example, encoded by a polymorphic gene whose sequence differs between the donor and recipient individuals.

Amino acid: As used herein, the term “amino acid,” in its broadest sense, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide.

Anaphylactic antigen: The phrase “anaphylactic antigen”, as used herein, refers to an antigen (e.g., an allergen) that is recognized to present a risk of anaphylactic reaction in allergic individuals when encountered in its natural state, under normal conditions. For example, pollens, animal danders or excretions (e.g., saliva, urine), certain food antigens, insect antigens, drugs, and rubber (e.g., latex) antigens are generally considered to be anaphylactic antigens. Exemplary anaphylactic antigens include those to which reactions are so severe as to create a risk of death (e.g., nuts, seeds, and fish).

Anaphylactic reaction: The phrase “anaphylactic reaction,” (e.g., “anaphylaxis”) as used herein, refers to a severe, whole body allergic reaction to an allergen, characterized by pathological responses in multiple target organs, e.g., airway, skin digestive tract, and cardiovascular system. As noted above, symptoms of severe allergic reactions such as anaphylactic reactions typically develop quickly, often within minutes of exposure to the allergen, and can include, for example, abdominal pain, abdominal breathing sounds (typically high-pitched), anxiety, chest discomfort or tightness, cough, diarrhea, difficulty breathing, difficulty swallowing, dizziness or light-headedness, flushing or redness of the face, nausea or vomiting, palpitations, swelling of the face, eyes or tongue, unconsciousness, wheezing, and combinations thereof. Particular signs of anaphylaxis may include, for example, abnormal heart rhythm (arrhythmia), fluid in the lungs (pulmonary edema), hives, low blood pressure, mental confusion, rapid pulse, skin that is blue from lack of oxygen or pale (e.g., from shock), swelling (angioedema) in the throat that may be severe enough to block the airway, swelling of the eyes and/or face, weakness, wheezing. The most severe anaphylactic reactions can result in loss of consciousness and/or death.

Animal: As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Antigen: The term “antigen”, as used herein, refers to an agent that elicits an immune response; and/or (ii) an agent that binds to a T cell receptor (e.g., when presented by an MHC molecule) or to an antibody (e.g., produced by a B cell). In some embodiments, an antigen elicits a humoral response (e.g., including production of antigen-specific antibodies); in some embodiments, an elicits a cellular response (e.g., involving T-cells whose receptors specifically interact with the antigen). In general, and antigen may be or include any chemical entity such as, for example, a small molecule, a nucleic acid, a polypeptide, a carbohydrate, a lipid, etc. In some embodiments, an antigen is or comprises a polypeptide. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present disclosure are provided in a crude form. In some embodiments, an antigen is a recombinant antigen.

Antigen presenting cell: The phrase “antigen presenting cell” or “APC,” as used herein, has its art understood meaning referring to cells which process and present antigens to T-cells. Exemplary antigen cells include dendritic cells, macrophages and certain activated epithelial cells.

Approximately: As used herein, the term “approximately” and “about” is intended to encompass normal statistical variation as would be understood by those of ordinary skill in the art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are “associated” with one another if they interact, directly or indirectly, so that they are and remain in physical proximity with one another.

Autoantigen: As used herein, the term “autoantigen” is used to refer to antigens produced by an individual that are recognized by the immune system of that individual. In some embodiments, an autoantigen is one whose recognition by the individual's immune system is associated with an autoimmune disease, disorder or condition. In general, an autoantigen may be or include any chemical entity such as, for example, a small molecule, a nucleic acid, a polypeptide, a carbohydrate, a lipid, etc. In some embodiments, an autoantigen is or comprises a polypeptide. Those of skill in the art are familiar with a variety of agents, including polypeptides, that can act as autoantigens, and particular that are recognized in immune reactions associated with autoimmunity diseases, disorders and/or conditions.

Biocompatible: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

Biodegradable: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Biologically active: As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a crude lysate in that little or no purification is performed after disruption of the cells, which generates a “primary” lysate. In some embodiments, one or more isolation or purification steps is performed on the primary lysate. However, the term “lysate” refers to a preparation that includes multiple cellular components and not to pure preparations of any individual component.

Combination therapy: As used herein, the term “combination therapy” refers to those situations in which a subject is simultaneously exposed to two or more therapeutic agents. In some embodiments, such agents are administered simultaneously; in some embodiments, such agents are administered sequentially; in some embodiments, such agents are administered in overlapping regimens.

Complex Mixtures: As used herein, the term “complex mixtures” refers to mixtures of a plurality of components in inexact proportions. In some embodiments, complex mixtures may comprise agents or entities of a plurality of different chemical classes (e.g., polypeptide, nucleic acids, lipids, carbohydrates, metals and/or combinations thereof). In some embodiments, a complex mixture may be or include agents and/or entities that are naturally found in a common source (e.g., in a cell or organism); in some such embodiments, such agents and/or entities may be present in the complex mixture in relative amounts comparable to those in which they are present in the source. In some embodiments, a complex mixture may include or represent a significant (e.g., more than about 20%, 25%, 30%, 35%, 40%, 45%, 50%, 55%, 60%, 65%, 70%, 75%, 80%, 85%, 90%, 95%, or 100%) fraction of components or materials found in a particular source.

Corresponding to: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190^{th} residue in the first polymer but rather corresponds to the residue found at the 190^{th} position in the reference polymer; those of ordinary skill in the art readily appreciate how to identify “corresponding” amino acids, including through use of one or more commercially-available algorithms specifically designed for polymer sequence comparisons.

Derivative: As used herein, the term “derivative” refers to a structural analogue substance that is produced or formed from another substance of similar structure in one or more steps. In some embodiments, a derivative refers to a second chemical substance related structurally to a first chemical substance and theoretically derivable from the first chemical substance. By way of example, cellulose derivatives include, but are not limited to, cellulose esters (such as organic and inorganic esters), cellulose ethers (such as alkyl, hydroxyalkyl and carboxyalkyl ethers), sodium carboxymethyl cellulose and cellulose acetate. One of skill in the art will be able to extrapolate derivatives of other molecules accordingly.

Dosage form: As used herein, the term “dosage form” refers to a physically discrete unit of a therapeutic agent for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is correlated with a desired or beneficial outcome when administered across a relevant population (i.e., is a therapeutic dosing regimen).

Encapsulated: The term “encapsulated” is used herein to refer to substances that are completely surrounded by another material.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” is used to refer to a form or fragment of an entity that exhibits a particular property and/or activity.

Graft rejection: The term “graft rejection” as used herein, refers to rejection of tissue transplanted from a donor individual to a recipient individual. In some embodiments, graft rejection refers to an allograft rejection, wherein the donor individual and recipient individual are of the same species. Typically, allograft rejection occurs when the donor tissue carries an alloantigen against which the recipient immune system mounts a rejection response. In some embodiments, graft rejection refers to a xenograft rejection, wherein the donor and recipient are of different species. Typically, xenograft rejection occurs when the donor species tissue carries a xenoantigen against which the recipient species immune system mounts a rejection response.

Host: The term “host” is used herein to refer to an organism that receives a transplant. In some embodiments, the host is of a different species than is the source of the donor tissue (e.g., than is the donor or the source from which cells of the donor tissue were derived). In some embodiments, the host is of the same species as is the source of the donor. In some embodiments, the host is the source of the donor tissue.

Host Cell: The term “host cell” as used herein, refers any host cell that can be used to express a product of interest (e.g., an antigen). The cells can be genetically engineered to contain a recombinant nucleic acid sequence, e.g., a gene, that encodes a product of interest (e.g., an antigen). Host cells may be naturally-derived, engineered, and/or combinations thereof.

Human: In some embodiments, a human is an embryo, a fetus, an infant, a child, a teenager, an adult, or a senior citizen.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent identity between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. Representative algorithms and computer programs useful in determining the percent identity between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined for example using the GAP program in the GCG software package using an NWSgapdna.CMP matrix.

Infection: As used herein, the term “infection” refers to the invasion of a host organism's body by a disease-causing organism that multiplies in the host. Symptoms of an infection may result from action of toxins produced by the disease-causing organism and/or be reaction of host tissues to the organisms and/or to toxins they produce.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Patient: As used herein, the term “patient” or “subject” refers to a human or any non-human animal (e.g., mouse, rat, rabbit, dog, cat, cattle, swine, sheep, horse or primate) to whom therapy is administered. In many embodiments, a patient is a human being. In some embodiments, a patient is a human presenting to a medical provider for diagnosis or treatment of a disease, disorder or condition. In some embodiments, a patient displays one or more symptoms or characteristics of a disease, disorder or condition. In some embodiments, a patient does not display any symptom or characteristic of a disease, disorder, or condition. In some embodiments, a patient is someone with one or more features characteristic of susceptibility to or risk of a disease, disorder, or condition.

Pharmaceutically acceptable: The term “pharmaceutically acceptable” as used herein, refers to agents that, within the scope of sound medical judgment, are suitable for use in contact with tissues of human beings and/or animals without excessive toxicity, irritation, allergic response, or other problem or complication, commensurate with a reasonable benefit/risk ratio.

Polypeptide: The term “polypeptide”, as used herein, generally has its art-recognized meaning of a polymer of at least three amino acids. In some embodiments, the term is used to refer to specific functional classes of polypeptides, such as, for example, autoantigen polypeptides, nicotinic acetylcholine receptor polypeptides, alloantigen polypeptides, etc. For each such class, the present specification provides several examples of amino acid sequences of known exemplary polypeptides within the class; in some embodiments, such known polypeptides are reference polypeptides for the class. In such embodiments, the term “polypeptide” refers to any member of the class that shows significant sequence homology or identity with a relevant reference polypeptide. In many embodiments, such member also shares significant activity with the reference polypeptide. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region, often including a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Refractory: As used herein, the term “refractory” refers to any subject that does not respond with an expected clinical efficacy following the administration of provided compositions as normally observed by practicing medical personnel.

Small molecule: As used herein, the term “small molecule” means a low molecular weight organic compound that may serve as an enzyme substrate or regulator of biological processes. In general, a “small molecule” is a molecule that is less than about 5 kilodaltons (kD) in size. In some embodiments, provided compositions may further include one or more small molecules. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, one or more small molecules are encapsulated within an article. In some embodiments, small molecules are non-polymeric. In some embodiments, in accordance with the present disclosure, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

Stable: The term “stable,” when applied to compositions herein, means that the compositions maintain one or more aspects of their physical structure over a period of time. In some embodiments, a stable provided composition is one for which a biologically relevant activity is maintained for a period of time under specified conditions. In some embodiments, the period of time is at least about one hour; in some embodiments the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. For example, if a provided composition is subjected to prolonged storage, temperature changes, and/or pH changes, and a majority of the ingredients in the composition maintain one or more properties (e.g., bioactivity), the composition is considered stable. In some embodiments, a stable composition is stable at ambient conditions. In some embodiments, a stable composition is stable under biologic conditions (i.e., 37° C. in phosphate buffered saline).

Subject: As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition has been diagnosed with and/or exhibits or has exhibited one or more symptoms or characteristics of the disease, disorder, or condition.

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from allergy, etc).

Symptoms are reduced: According to the present disclosure, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. In some embodiments, a strategy may be considered to prolong or delay based on a comparison with an appropriate reference such as, for example, a) no treatment, or b) treatment with the current standard of care.

Th1-associated antigen: As used herein, the phrase “Th1-associated antigen” refers to any antigen typically associated with Th1 responses. Such an antigen would typically cause a predominantly Th1 immune response in a subject upon exposure of that subject to the antigen.

Th2-associated antigen: As used herein, the phrase “Th2-associated antigen” refers to any antigen typically associated with Th2 responses. Such an antigen would typically cause a predominantly Th2 immune response in a subject upon exposure of that subject to the antigen.

Therapeutic agent: As used herein, the phrase “therapeutic agent” refers to any agent that has a therapeutic effect and/or elicits a desired biological and/or pharmacological effect, when administered to a subject. In some embodiments, an agent is considered to be a therapeutic agent if its administration to a relevant population is statistically correlated with a desired or beneficial therapeutic outcome in the population, whether or not a particular subject to whom the agent is administered experiences the desired or beneficial therapeutic outcome.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition (e.g., allergy). In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective agent may be formulated and/or administered in a single dose. In some embodiments, a therapeutically effective agent may be formulated and/or administered in a plurality of doses, for example, as part of a dosing regimen.

Therapeutic regimen: A “therapeutic regimen”, as that term is used herein, refers to a dosing regimen whose administration across a relevant population is correlated with a desired or beneficial therapeutic outcome.

Treatment: As used herein, the term “treatment” (also “treat” or “treating”) refers to any administration of a substance that partially or completely alleviates, ameliorates, relives, inhibits, delays onset of, reduces severity of, and/or reduces frequency, incidence or severity of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Undesirable Immune Response: As used herein, the phrase “undesirable immune response” refers to lack of an effective immune response (e.g., such as for cancer).

### DETAILED DESCRIPTION

The present disclosure is based, in part, on the insight that immunomodulation may be accomplished via crude preparations of antigens and/or of adjuvant agents. In some embodiments, these preparations may comprise antigens and/or adjuvants that are be co-localized. In some embodiments these preparations are useful for treatment, e.g. of certain diseases, disorders or conditions. In some embodiments, such immune modulation may comprise or consist of induction, stimulation, and/or maintenance of a type of response (e.g., Th1, Th2, Treg, T0, Th17, etc) not usually associated with the antigen.

The present disclosure is based, in part, on the insight that undesirable immune responses, for example, anaphylaxis, may be mitigated or even prevented through the use of at least one antigen (e.g., at least one Th2-associated antigen) in combination with at least one adjuvant agent (e.g., at least one crude adjuvant agent). In some embodiments, administration of such a composition results in an immune response that is skewed from a predominantly Th2 response to a response that is not predominantly Th2 (e.g., a predominantly Th1 and/or Treg response). In accordance with various embodiments, provided methods and compositions may include at least one crude Th2-associated antigen and/or at least one crude adjuvant agent. In some embodiments, the present invention provides compositions including at least one antigen substantially co-localized with at least one adjuvant agent in a format selected from patch, capsule, tablet, gel, matrix, paste, reservoir, adhesive, liquid, suspension, lyophilized solid, liquid contained within one or more lumenal units, and combinations thereof, wherein one or both of the antigen and the adjuvant agent is a crude preparation, and wherein the composition is characterized in that administration to a subject in need thereof results in an immune response in the subject to the antigen. In some embodiments, the antigen is a Th2-associated antigen and the immune response is not a predominantly Th2 response. In some embodiments, the antigen is a Th1-associated antigen and the immune response is not predominantly Th1 response. In some embodiments, provided compositions and/or methods do not include and/or do not utilize nanoparticles and/or microparticles.

The Immune System

The immune system is an extraordinarily complex system of interrelated signaling pathways and cellular responses responsible for maintaining the integrity of a subject's body. The immune system is responsible for, inter alia, preventing and/or fighting disease and responding to injury (e.g., trauma). Immunity is generally categorized as adaptive or innate, with the adaptive immune system generally being specific for particular pathogens and able to provide long-lasting protection, whereas the innate immune system tends to be less specific, defending the body against, e.g., foreign organisms. Though the adaptive immune system may destroy pathogens, it may also have difficulty distinguishing between pathogens and non-pathogens, resulting in conditions such as, for example, allergies. The adaptive immune system is further divided into humoral and cell-mediated responses, driven by B lymphocytes (e.g., production of immunoglobulins) and T lymphocytes (associated with, e.g., cell-mediated cytotoxicity), respectively. Immune responses and effects are largely mediated, in whole or in part, by cytokines. Cytokines, which represent a well-studied category of small, secreted proteins and play important roles in cell signaling, can be released by T lymphocytes. Cytokines are responsible for many of the biological effects of the immune system.

T lymphocytes are a major source of many cytokines. T cells include antigen specific receptors on their surface to facilitate recognition of foreign pathogens in the body by distinguishing between “self” and “non-self”. There are two main categories of T cells, which are classified by the presence of either CD4 or CD8 cell surface molecules. Generally, T cells expressing CD8 are characterized as cytotoxic T cells, while those expressing CD4 are known as helper T (“Th”) cells, and are the largest producers of cytokines in the body. Th cells differentiate from progenitors into effector, memory or regulatory Th cell phenotypes. Effector Th cells may be further classified into two major subtypes: Th1 or Th2, which is largely based on the type of cytokines they secrete. Since Th1 and Th2-responses are generally reciprocally inhibitory to one another, it is often important that they normally exist and respond (when necessary) in a balance. When easily or chronically biased towards one phenotype or another, either as a baseline, or in response to an event, dysfunction (such as, e.g., autoimmunity or anaphylaxis) may result.

The Immune System—Th1 Responses

Th1 cells are important for cellular immune responses and host defense from intracellular pathogens, secreting pro-inflammatory cytokines which cause macrophages and other types of lymphocytes to phagocytose microbial pathogens. Th1-type, pro-inflammatory cytokines include, e.g., IFN-γ, IL-2, TNF-β, and/or IL-12, and/or immunoglobulins such as IgG2. Such Th1-type cytokines are, inter alia, responsible for killing intracellular pathogens (e.g., parasites, which may include viruses and/or bacteria), and can also underlie autoimmunity. Uncontrolled and/or excessive Th-1/pro-inflammatory responses can result in significant tissue damage. A Th1 response may be or comprise a variety of specific immune responses. A Th1 response may be characterized by decreased activity of one or more of IL-4, IL-5, IL-6, IL-9, IL-10, IL-13, IgE, and histamines relative to administration of a Th2-associated antigen to the subject without at least one adjuvant agent. A Th1 response may be characterized by increased activity of one or more of IFN-γ, IL-2, TNF-β, IL-12, and IgG2a relative to administration of a Th2-associated antigen to the subject without at least one adjuvant agent.

The Immune System—Th2 Responses

Th2 cells are important for humoral immunity. These cells respond by secreting cytokines, protecting against, e.g., extracellular parasites. Th2-type cytokines include interleukins 4, 5, 10 and 13, and stimulate production of antibodies. Th2-type cytokines and responses are associated generally with atopy and allergy, as well as the promotion of IgE-mediated and eosinophilic responses and general anti-inflammatory effects. It is known that, in excess, Th2-type cytokines are able to counteract Th1 responses, which, in some situations, may be detrimental to the host organism. It is also known that Th2 responses are responsible for many of the symptoms and effects of allergy which, in extreme cases, may include anaphylactic responses.

A Th2 response may be or comprise a variety of specific immune responses. A Th2 response may be characterized by increased activity of one or more of IL-4, IL-5, IL-6, IL-9, IL-10, IL-13, IgE, and histamines relative to administration of a Th1-associated antigen to the subject without at least one adjuvant agent. A Th2 response may be characterized by decreased activity of one or more of IFN-γ, IL-2, TNF-β, IL-12, and IgG2a relative to administration of a Th1-associated antigen to the subject without at least one adjuvant agent.

The Immune System—T_{reg} Responses

Regulatory T cells, also known as T_{reg} or suppressor T cells, are a subpopulation of T cells which modulate the immune system by decreasing immune function, which allows for tolerance to self-antigens. Normal function of T_{reg} cells may inhibit or prevent autoimmunity from developing, while dysfunction of Tregs are known to play a role in development of autoimmune disease, transplant rejection, and even cancer. In general, T_{reg} function to suppress the activity of other immune cells in a subject's body. Specific subpopulations of T_{reg} are known to exist including CD4+, CD25+, and Foxp3+T_{reg}.

A T_{reg} response may be characterized by a variety of specific immune responses.

The Immune System—Th17 Responses

Th17 cells may be characterized by their production of interleukin-17 (IL-17) and lack of production of classical Th1 and/or Th2 cytokines, such as IFN-γ or IL-4. Th17 cells comprise a distinct lineage of T cell, which functions to modulate host defense against extracellular (e.g. fungal and bacterial) pathogens. Th17 cells are also implicated in induction and perpetuation of pro-inflammatory, and autoimmune reactions. In some cases, Th17 responses may comprise amplification of acute inflammation at sites of infection.

In some embodiments, administration of a provided composition results in a Th17 response in a subject. According to various embodiments, a Th17 response may be or comprise a variety of specific immune responses.

### Antigens

Any of a variety of antigens may be used in accordance with various embodiments. It will be appreciated by those skilled in the art that any entity to which an immune reaction can be directed may be an antigen. The present disclosure provides, generally, technologies capable of modulating immune responses to antigens. In some embodiments, such immune modulation may comprise or consist of induction, stimulation, and/or maintenance of a type of response (e.g., Th1, Th2, T_{reg}, Th17, etc) not usually associated with the antigen.

In some embodiments, an antigen is or comprises an allergic antigen. In some embodiments, an antigen is or comprises an anaphylactic antigen. In some embodiments, an antigen is or comprises an infectious antigen. In some embodiments, an antigen is or comprises an autoantigen. In some embodiments, an antigen is or comprises a disease-associated antigen.

In some embodiments, an antigen is or comprises an infectious agent antigen. In some embodiments, an infectious antigen is provided with one or more additional components of an infectious agent. In some embodiments, an infectious agent is selected from the group consisting of a second infectious antigen, a disease-associated antigen (e.g. a tumor-associated antigen), an autoantigen, an allergen, or combinations thereof.

In some embodiments, provided compositions include at least one antigen. An antigen may be derived from any source, including, but not limited to, a virus, bacterium, parasite, plant, protozoan, fungus, tissue or transformed cell such as a cancer or leukemic cell and can be a whole cell or immunogenic component thereof, e.g., cell wall components or molecular components thereof. In some embodiments, crude extracts including one or more antigens may be used. In some embodiments, provided methods and/or compositions do not include and/or do not utilize substantially pure antigens.

In some embodiments, suitable antigens are known in the art and are available from commercial, government, and/or scientific sources. In some embodiments, antigens are provided from whole inactivated or attenuated organisms. In some embodiments, antigens are provided from organisms engineered to express one or more antigens. In some embodiments, antigens may be provided from infectious organisms, such as viruses, parasites and bacteria. In some embodiments, antigens may be provided from tumor cells. In some embodiments, antigens may be purified or partially purified polypeptides derived from tumors or viral or bacterial sources.

In some embodiments, antigens may be provided as single antigens or may be provided in combination (e.g., two or more antigens).

In some embodiments, antigens may be provided as complex mixtures.

In some embodiments, antigens may be recombinant polypeptides produced by expressing DNA encoding the polypeptide antigen in a heterologous expression system. In some embodiments, antigens can be DNA encoding all or part of an antigenic protein. In some embodiments, DNA may be in the form of vector DNA such as plasmid DNA.

In some embodiments, antigens are provided as a crude extract. In some embodiments, provided compositions may include one or more crude antigenic extracts. In some embodiments, a crude extract can be a useful and inexpensive alternative to using individual antigens in provided compositions.

In some embodiments, provided compositions may include one or more viral antigens. Generally, a virus consists of either two or three parts: 1) genetic material, which may be DNA or RNA, depending on the virus, 2) a protein coat that surrounds and protects the genetic material, and, in some viruses, 3) a lipid envelope that surrounds the protein coat. In some embodiments, a viral antigen may be provided from any component of a virus. In some embodiments, a viral antigen may be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronavtridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenza virus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D 1 NS 1, Dengue D 1 NS2, and Dengue D1NS3. In some embodiments, a viral antigen may comprise or consist of fragments of one or more viruses, such as fragments from an influenza virus, for example. In some embodiments, viral fragments are provided from one or more of 1) viral genetic material 2) a portion of a viral protein coat, and/or 3) a portion of a viral lipid envelope. In some embodiments, viral fragments may be provided from two or more of 1) viral genetic material 2) a portion of a viral protein coat, and/or 3) a portion of a viral lipid envelope.

Exemplary viral antigens include, but are not limited to, those found in the following viral strains such as an adenovirus, borrelia, chagas, coxsackieviruses, cytomegalovirus, dengue, Epstein-Barr (EBV), encephalitis (e.g., equine encephalitis and Japanese encephalitis), hantavirus, hepatitis A (HAV), hepatits B (HBV), hepatitis C (HCV), delta hepatitis D (HDV), hepatitis E (HEV), hepatitis G virus (HGV), herpes simplex virus (HSV)(i.e. HSV1 and HSV2), human immunodeficiency virus (HIV), human T-lymphotrophic virus (HTLV), influenza, lymphocytic choriomeningitis (LCMV), malaria, measles, mycoplasma, papillomavirus (e.g., human papillomavirus, HPV), parainfluenza, parvovirus, rhinovirus, Rift Valley fever, rotavirus, rubella, SARS, toxoplasma, treponema, varicella-zoster (VZV), west nile virus (WNV), yellow fever, and combinations thereof.

In some embodiments, provided compositions may include one or more bacterial antigens. Bacterial antigens may originate from any bacteria including, but not limited to Actinomyces, Aeromonas, Anabaena, Arthrobacter, Bacillus, Bacteroides, Bdellovibrio, Bordetella, Borrelia, Campylobacter, Caulobacter, Chlamydia, Chlorobium, Chromatium, Citrobacter, Clostridium, Corynebacterium, Cytophaga, Deinococcus, Enterobacter, Escherichia, Francisella, Haemophilus, Halobacterium, Heliobacter, Hemophilus influenza type B (HIB), Hyphomicrobium, Klebsiella, Lactococcus, Legionella, Leptspirosis, Listeria, Meningococcus A, B and C, Methanobacterium, Micrococcus, Morganella, Mycoplasma, Myobacterium, Myxococcus, Neisseria, Nitrobacter, Oscillatoria, Peptococcus, Phodospirillum, Plesiomonas, Prochloron, Proteus, Providencia, Pseudomonas, Rickettsia, Salmonella, Serratia, Shigella, Spirillum, Spirochaeta, Sporolactobacillu, Staphylococcus, Streptococcus, Streptomyces, Sulfolobus, Thermoplasma, Thiobacillus, Treponema, Vibrio, Yersinia, and combinations thereof.

In some embodiments, provided compositions may include one or more parasite antigens. Parasite antigens can be obtained from parasites such as, but not limited to, an antigen derived from Candida albicans, Candida tropicalis, Chlamydia trachomatis, Chlamydial psittaci, Cryptococcus neoformans, Entamoeba histolytica, Histoplasma capsulatum, Mycoplasma pneumoniae, Nocardia asteroides, Plasmodium falciparum, Rickettsia ricketsii, Rickettsia typhi, Schistosoma mansoni, Toxoplasma gondii, Trichomonas vaginalis and Trypanosoma brucei. These include Sporozoan antigens, Plasmodian antigens, such as all or part of a Circumsporozoite protein, a Sporozoite surface protein, a liver stage antigen, an apical membrane associated protein, or a Merozoite surface protein.

In some embodiments, provided compositions may include one or more environmental antigens. Exemplary environmental antigens include, but are not limited to, those derived from naturally occurring allergens such as pollen allergens (tree-, weed-, and grass pollen allergens), insect allergens (inhalant, saliva and venom allergens), animal hair and/or dander allergens.

In some embodiments, an antigen (e.g., a Th2-associated antigen) may be or comprise an allergen found in certain foods, venom, drugs or rubber that are capable of eliciting allergic responses, and in particular anaphylactic allergic responses in an individual. Exemplary allergens that may induce anaphylaxis, include several protein allergens found in food (peanut, milk, egg, wheat), insect venom (i.e., bees, reptiles), drugs, and latex. In some embodiments, an environmental antigen may be one or more venoms. Stings from organisms that inject venoms, such as insect stings are known to cause anaphylaxis in individuals with allergies to the venom. In general, insect venom includes venom from Hymenoptera such as bees, hornets, wasps, yellow jackets, velvet ants, and fire ants. For example, venom from honey bees of the genus Apis can cause anaphylaxis in stung victims who are allergic (Weber et al. Allergy 42:464-470). The venom from honey bees contains numerous compounds which have been extensively studied and characterized (see for a reference, Banks and Shipolini. Chemistry and Pharmacology of Honey-bee Venom. Chapter 7 of Venoms of the Hymenoptera. Ed. T. Piek. Academic Press. London. 1986). The two main components of bee venom are phospholipase A2 and melittin and may be used in some embodiments for treating and preventing allergies to bee venom. Non-limiting examples of protein allergens found in food include proteins found in nuts (e.g., peanut, walnut, almond, pecan, cashew, hazelnut, pistachio, pine nut, brazil nut), seafood (e.g. shrimp, crab, lobster, clams), fruit (e.g. plums, peaches, nectarines; Ann Allergy Asthma Immunol 7(6):504-8 (1996); cherries, Allergy 51(10):756-7 (1996)), seeds (sesame, poppy, mustard), and soy and dairy products (e.g., egg, milk).

In some embodiments, protein antigens found in pollen-related food allergies may be used (e.g., birch pollen related to apple allergies). Important pollen allergens from trees, grasses and herbs originate from the taxonomic orders of Fagales, Oleales, Pinales and platanaceae including i.a., birch (Betula), alder (Alnus), hazel (Corylus), hornbeam (Carpinus) and olive (Olea), cedar (Cryptomeriaand juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including, e.g., herbs of the genera Ambrosia, Artemisia, and Parietaria.

In some embodiments, an antigen may be or comprise one or more allergens from house dust mites of the genus Dermatophagoides and Euroglyphus, storage mite, e.g Lepidoglyphys, Glycyphagus and Tyrophagus, cockroaches, midges and fleas e.g. Blatella, Periplaneta, Chironomus and Ctenocepphalides, mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

In some embodiments, it may be desirable to work in systems in which a single compound (e.g., a single protein) is responsible for an observed allergy. In some embodiments, an antigen may comprise more complex allergens and/or crude allergenic extracts. Therefore, in some embodiments, collections of more than one antigen may be used so that immune responses to multiple antigens may be modulated with a single embodiment. In some embodiments, two or more antigens (e.g., 3, 4, 5, 6, 7, 8, 9, 10 or more) may be used in a particular composition and/or method.

One of skill in the art will recognize that multiple antigenic molecules (e.g., Th2-associated antigens) may be delivered by provided compositions simultaneously and/or sequentially in accordance with methods of the present disclosure. Without limitation, multiple different antigenic molecules for one antigenic protein may be delivered, and/or multiple different antigenic molecules from different antigenic proteins may also be delivered. Further, multiple antigenic polypeptides and proteins may be delivered in accordance with various embodiments of the present disclosure. It is also recognized that single or multiple antigenic polypeptides and single or multiple cytokines may be delivered to individuals by provided compositions in accordance with the present disclosure. For example, but without limitation, allergenic antigens of the present disclosure and immunomodulatory molecules such as interleukins may be delivered by provided compositions using methods in accordance with the present disclosure. In some embodiments, provided methods and/or compositions do not include and/or do nut utilize nanoparticles and/or microparticles.

Those skilled in the art will further appreciate that certain antigens are typically associated with Th1 and/or Th2 responses.

In some embodiments, an antigen may be or comprise a Th2-associated antigen, either in general for humans or in a particular subject, or group of subjects. In some embodiments, a Th2 response may be or comprise an increase in at least one Th2-associated cytokine (e.g., IL-4, IL-5, IL-6, IL-9, IL-10, IL-13, IgE, and histamines, among others). In some embodiments, a Th2 response may be or comprise a decrease in one or more Th1-associated cytokines (e.g., IFN-γ, IL-2, TNF-β, IL-12, and IgG2a, among others).

In some embodiments, an antigen may be or comprise a Th1-associated antigen, either in general for humans or in a particular subject, or group of subjects. In some embodiments, a Th1 response may be or comprise an increase in at least one Th1-associated cytokine (e.g., IFN-γ, IL-2, TNF-β, IL-12, and IgG2a, among others). In some embodiments, a Th1 response may be or comprise a decrease in one or more Th2-associated cytokines (e.g., IL-4, IL-5, IL-6, IL-9, IL-10, IL-13, IgE, and histamines, among others).

Exemplary criteria for identifying and selecting effective Th2-associated antigenic peptides (e.g., minimal peptide sequences capable of activating a Th2 response in a subject) may be found in the art. For example, Apostolopoulos, et al. (Curr. Opin. Mol. Ther., 2:29-36 (2000)), discusses the strategy for identifying minimal antigenic peptide sequences based on an understanding of the three dimensional structure of an antigen-presenting molecule and its interaction with both an antigenic peptide and T-cell receptor. Shastri, (Curr. Opin. Immunol., 8:271-7 (1996)), discloses how to distinguish rare peptides that serve to activate T cells from the thousands peptides normally bound to MHC molecules.

The present disclosure encompasses the recognition that a particular subject may benefit from being exposed to a combination of antigens, such as multiple allergens. In some embodiments, it may be desirable to provide a composition comprising multiple antigens relevant to a specific subject, and/or to a population of subjects. For example, in some embodiments, a particular provided composition will contain a combination of allergens to address some or all of a particular subject's allergies and/or a combination of allergens to address some or all allergies commonly present within a population. For example, if a particular subject is allergic to peanuts and to dust mites, a composition may be designed and manufactured to address both allergies. Alternatively or additionally, in some embodiments it may be desirable to prepare compositions including antigens from a plurality of allergens (i) to which members of a particular community are commonly exposed (e.g., by virtue of geographic location); (ii) to which subjects are exposed by a common route (e.g., inhalation, ingestion, contact, etc); (iii) to which incidence of allergy within a relevant population (e.g., a geographic population, an age population, an ethnic population, etc) is above a designated threshold; (iv) to which subjects allergic to one allergen also tend to have allergy to, for example, subjects allergic to tree nuts tend to also be allergic to pecans, walnuts, and pistachios, subjects with allergy to crustaceans (e.g., lobster, crab, shrimp, or crayfish) or mollusks (e.g., clams, mussels, oysters, or scallops) tend to have allergy to various types, not just a single crustacean or mollusk. In some embodiments, provided methods and/or compositions do not include and/or do not utilize substantially pure antigens.

In some embodiments, a particular provided composition may contain a combination of Th2-associated antigens other than allergens. For example, in some embodiments, a particular provided composition may contain a combination of antigens associated with a particular disease, disorder or condition (e.g., with a particular cancer, a particular infectious disease, a particular graft vs host or host vs graft syndrome, etc).

Those of skill in the art will recognize a wide variety of potential applications utilizing combinations of Th2-associated antigens; each of these is contemplated as within the scope of the present disclosure.

According to various embodiments, provided compositions comprising a Th2-associated antigen or other Th2-associated protein may comprise the antigen or other protein in any of a variety of forms. Exemplary forms include, without limitation, RNA, DNA, protein, and combinations thereof. In some embodiments, the antigen or protein may be provided in a crude form, such as, for example, as a portion of a cell, tissue or extract thereof. In some embodiments, the antigen or protein is not substantially pure.

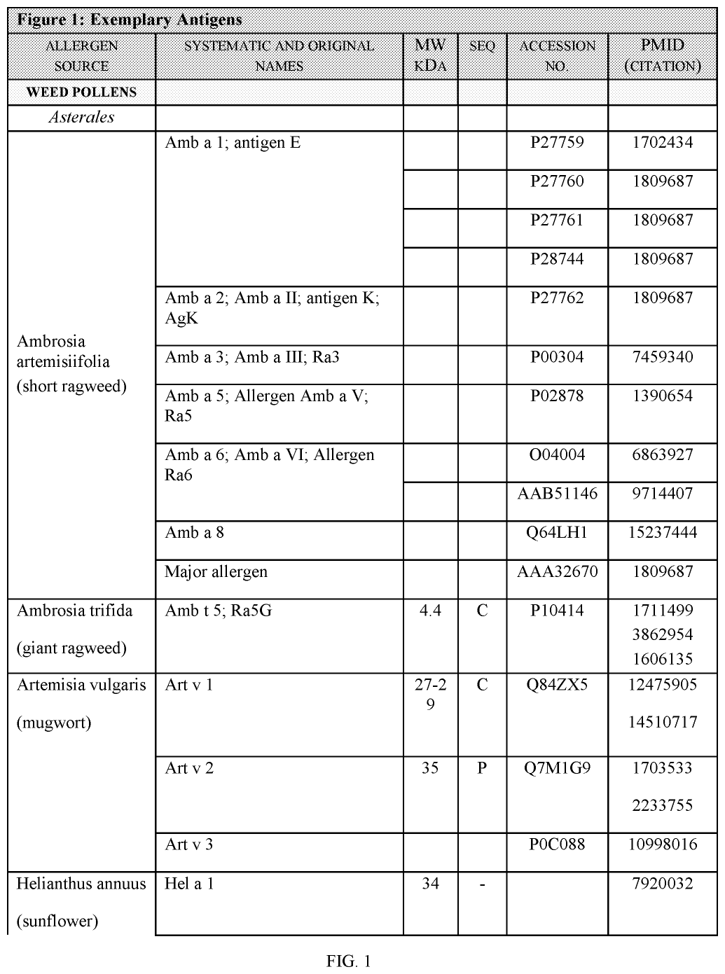

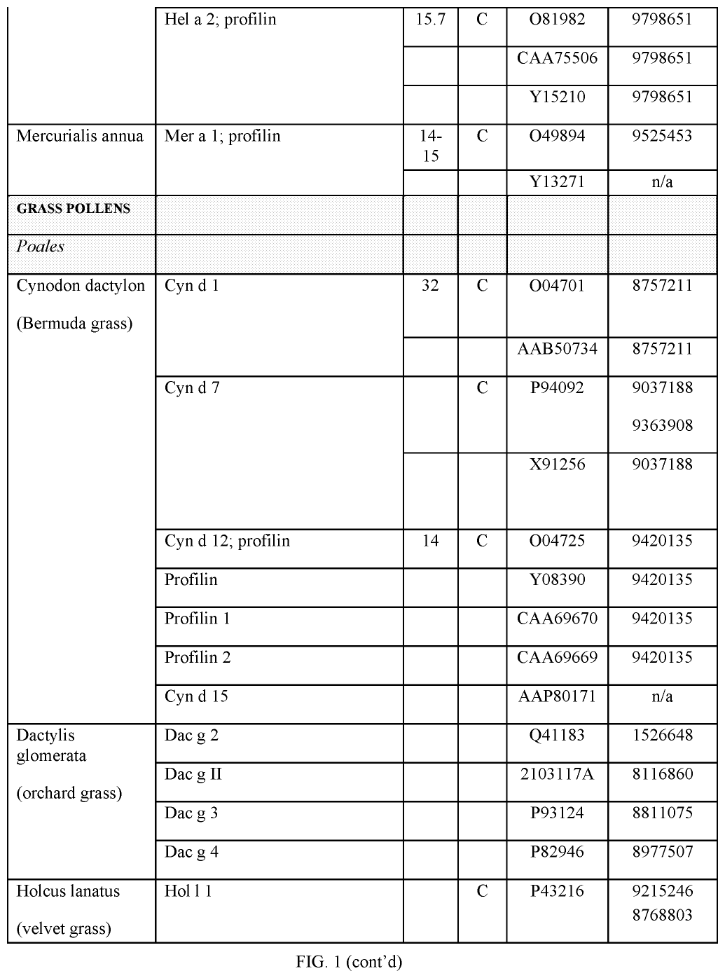

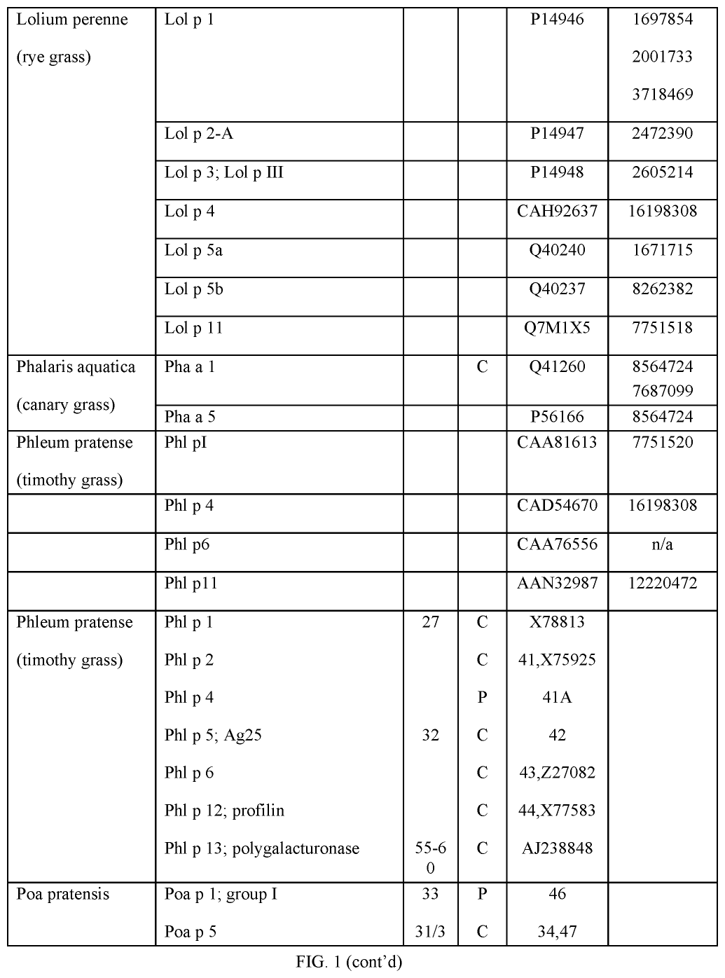

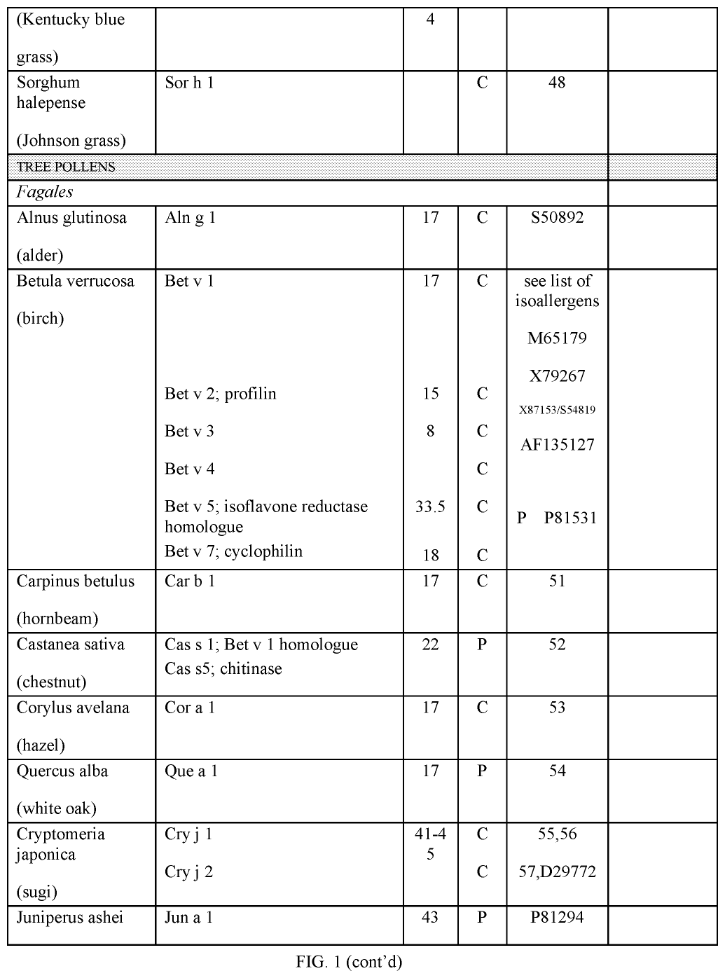

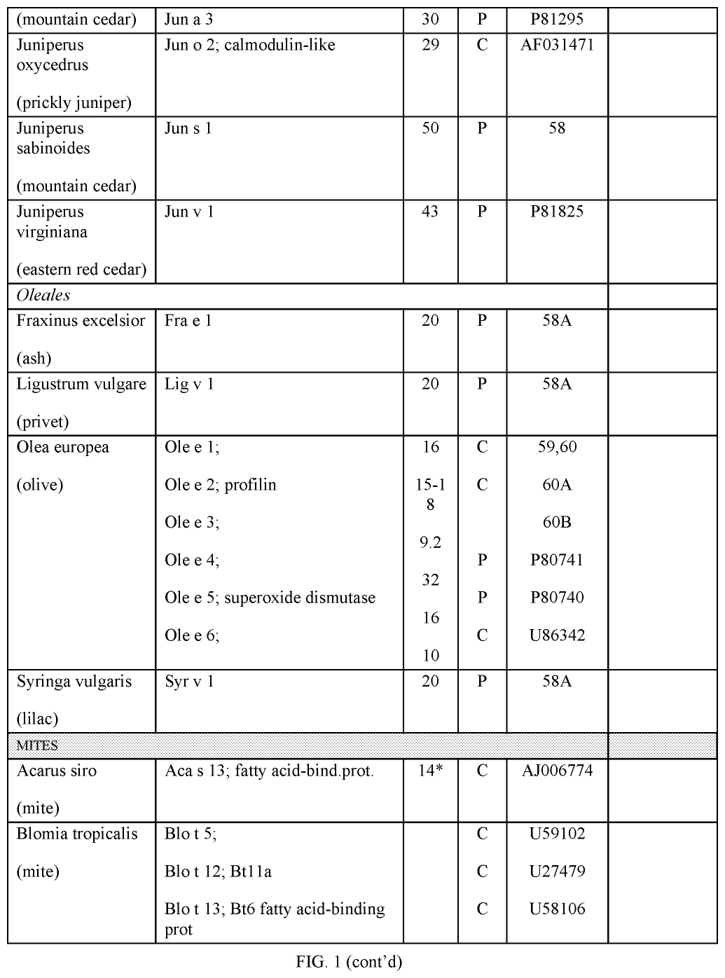

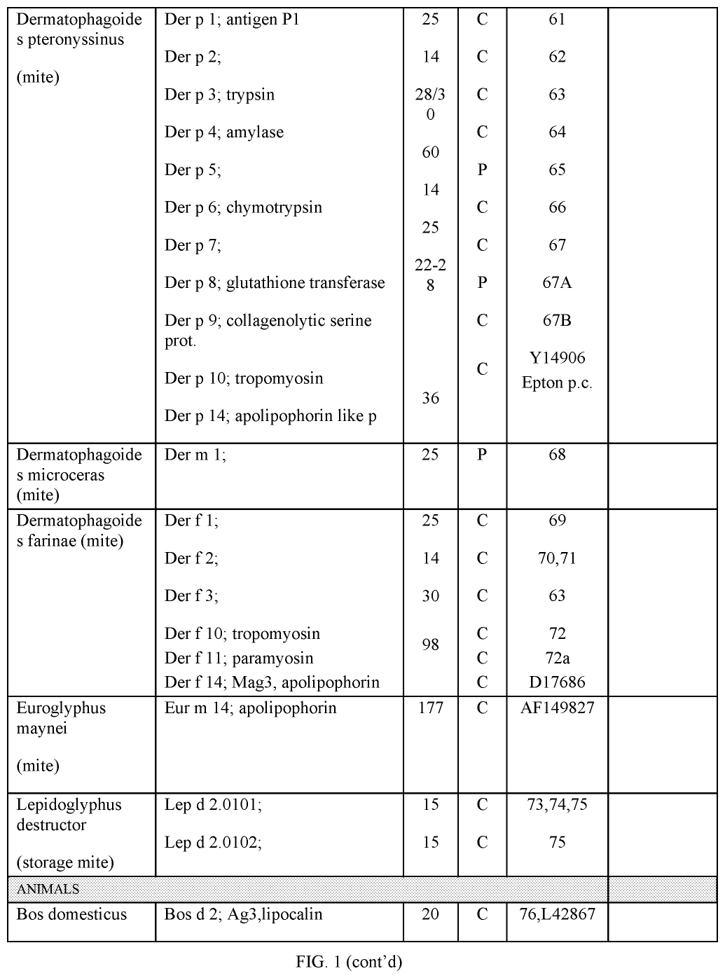

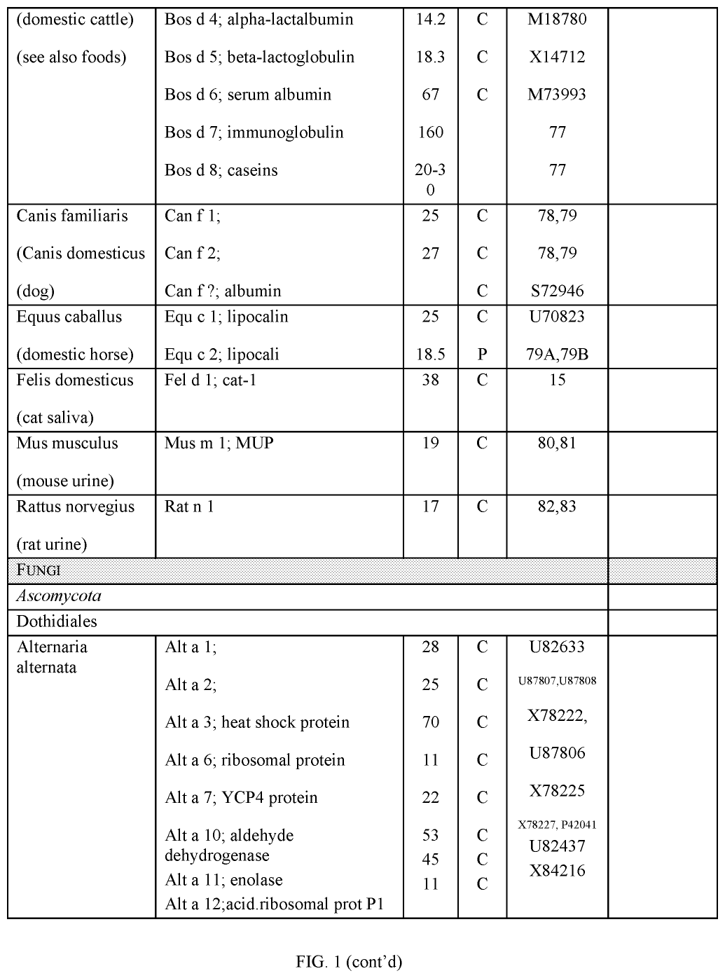

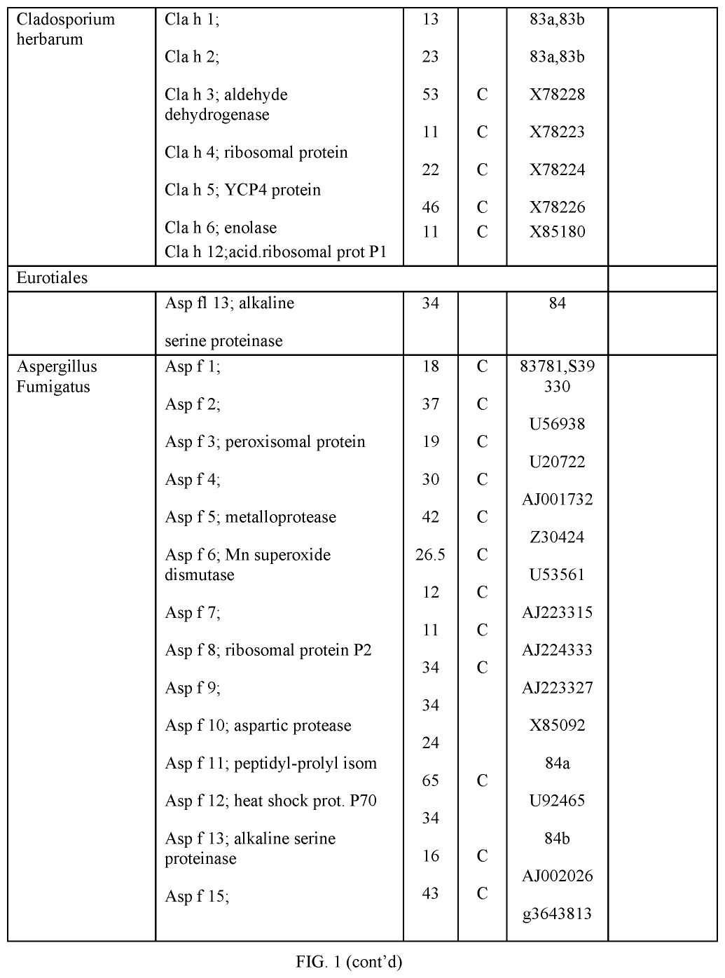

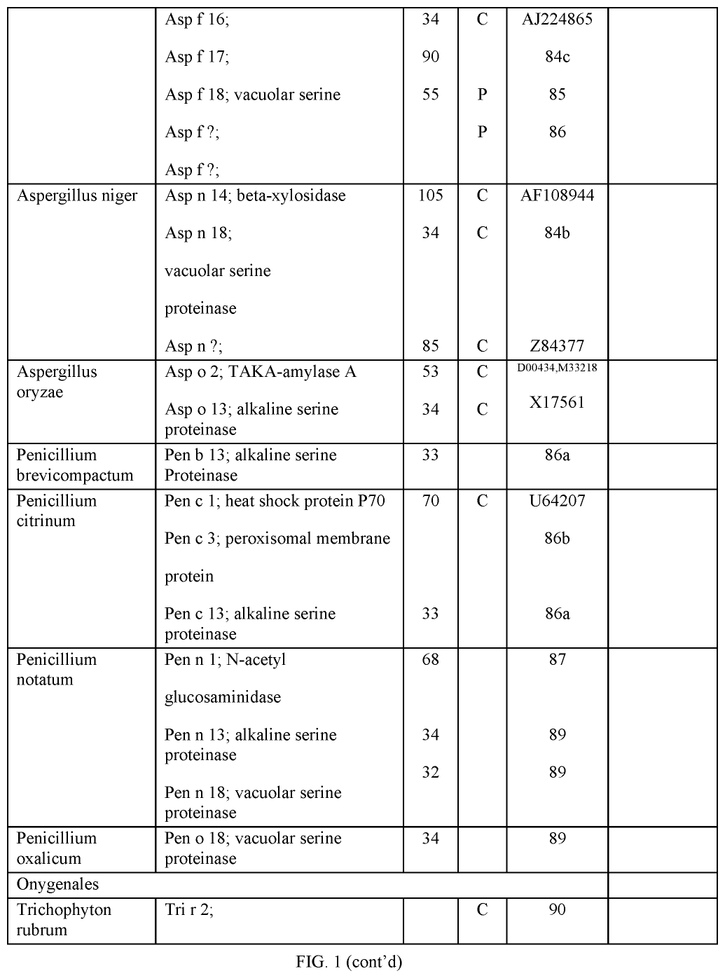

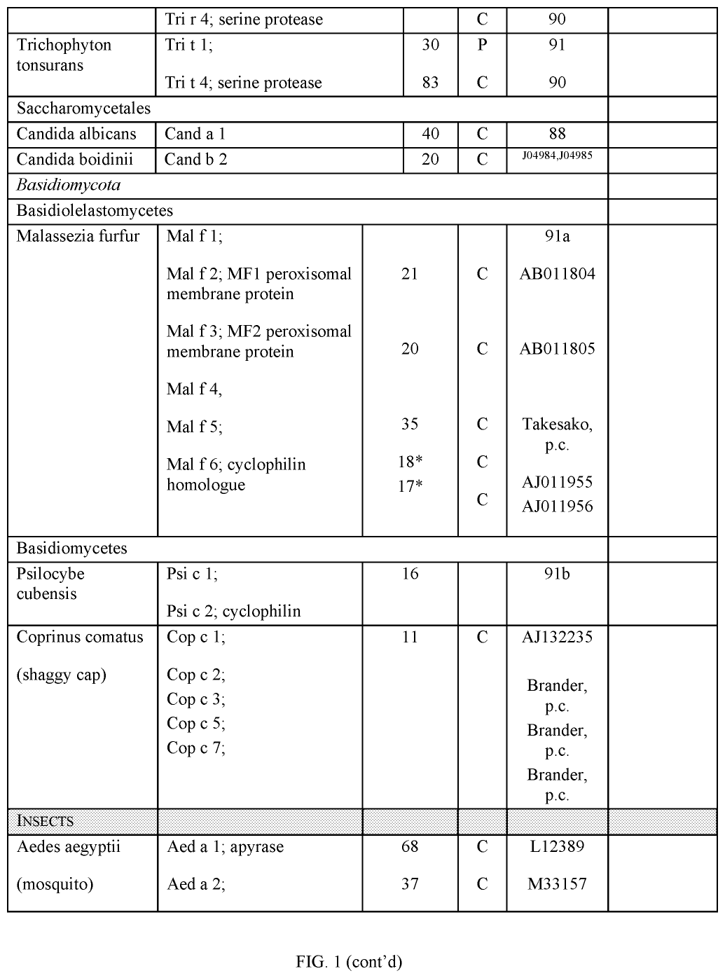

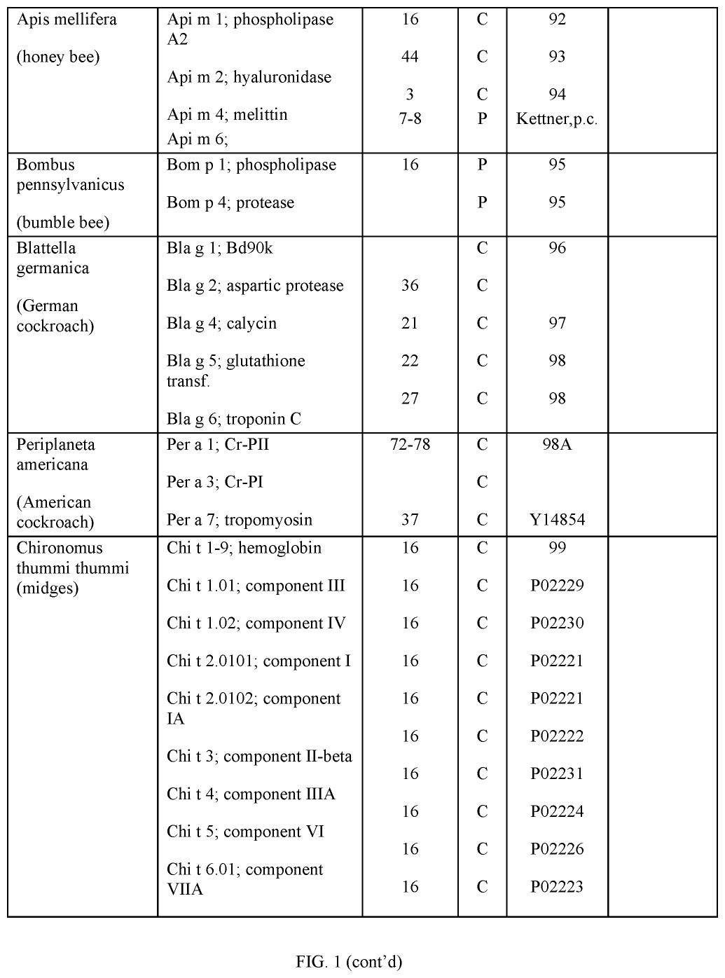

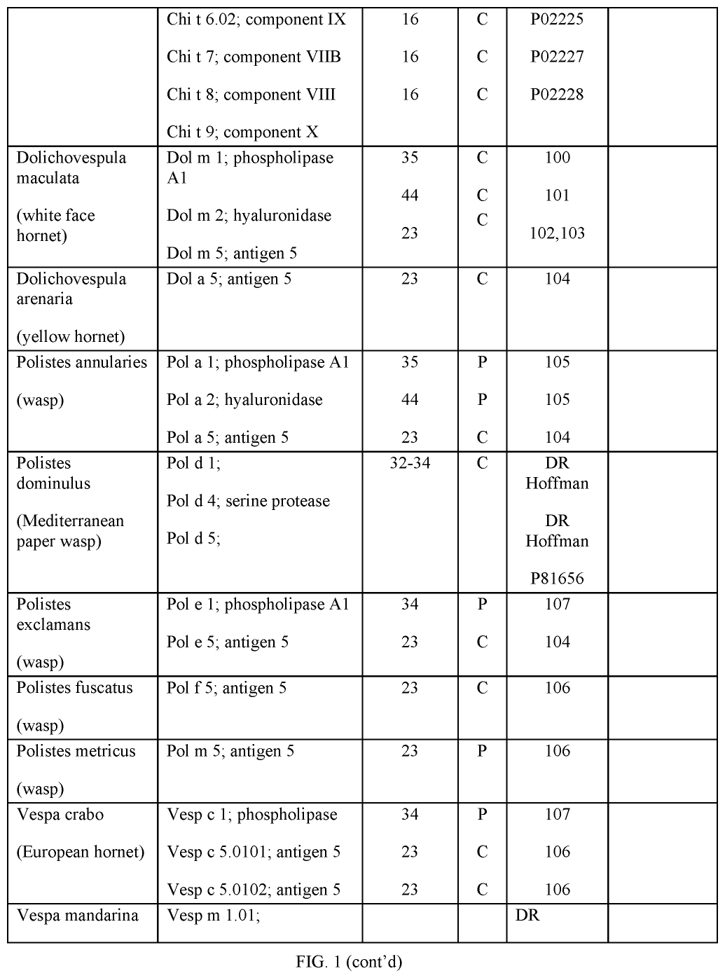

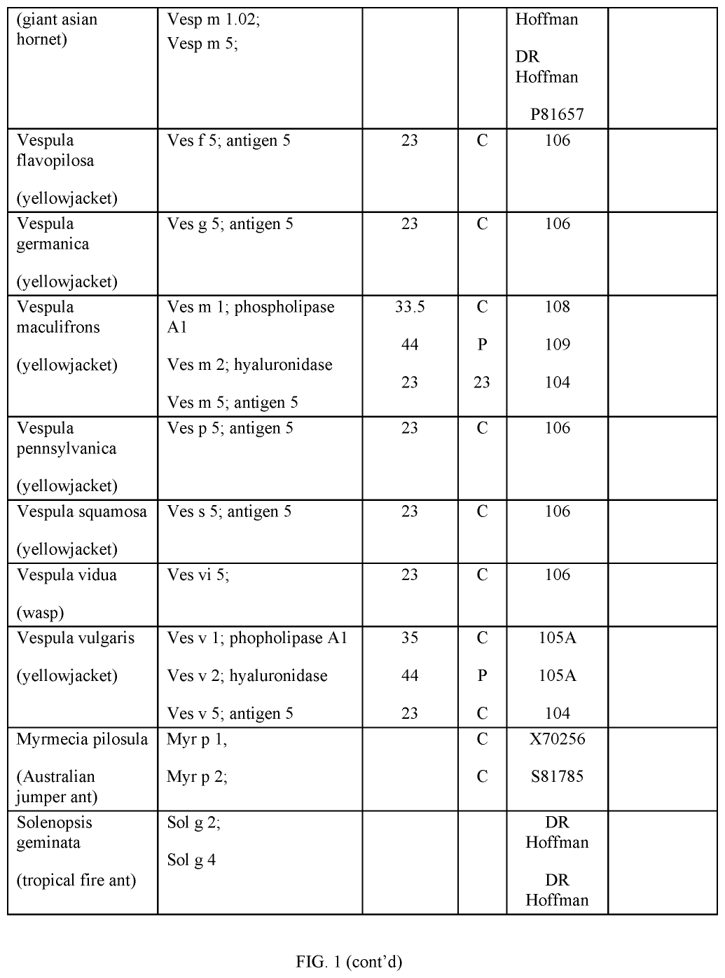

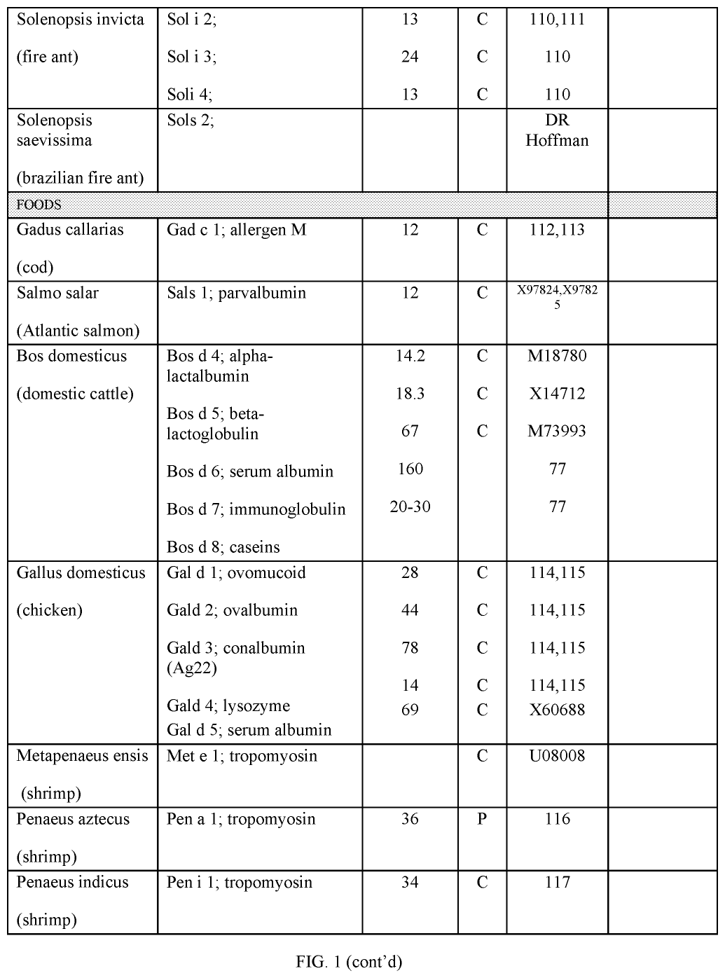

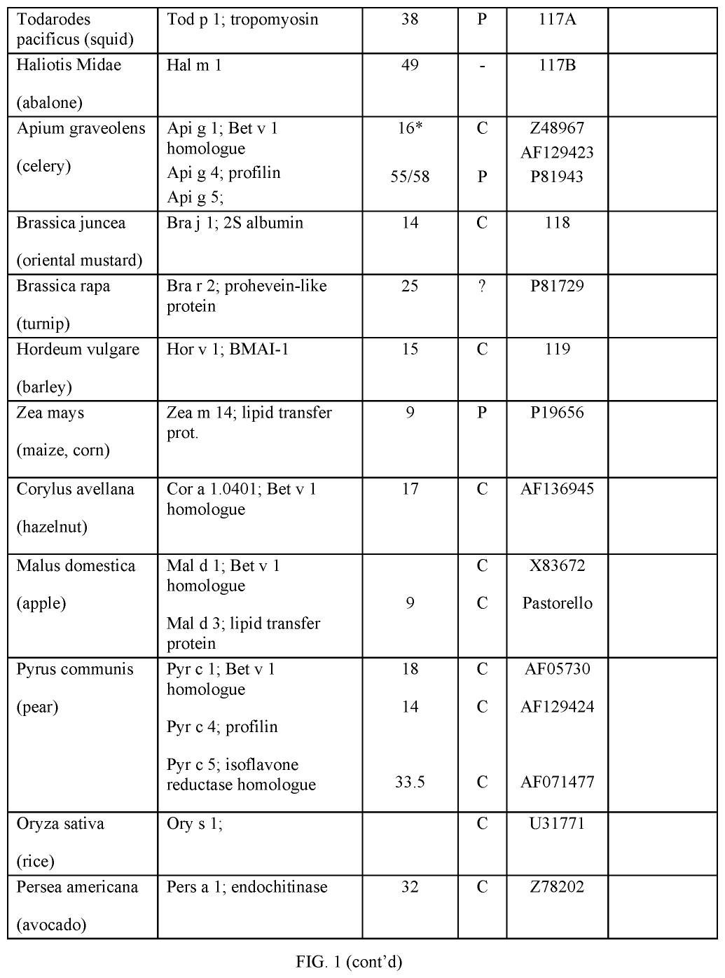

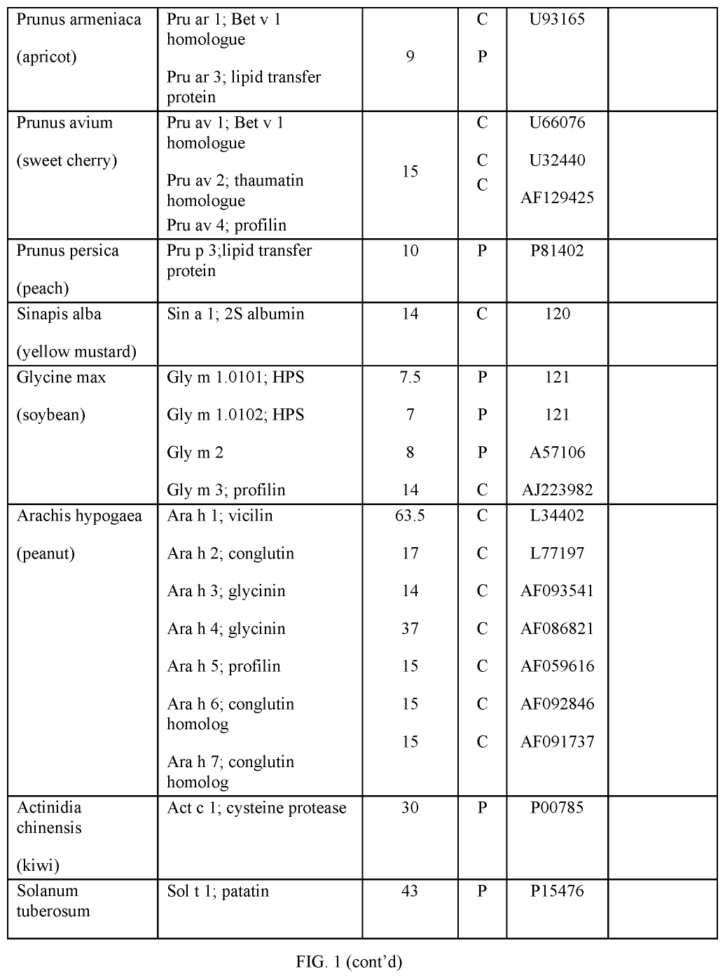

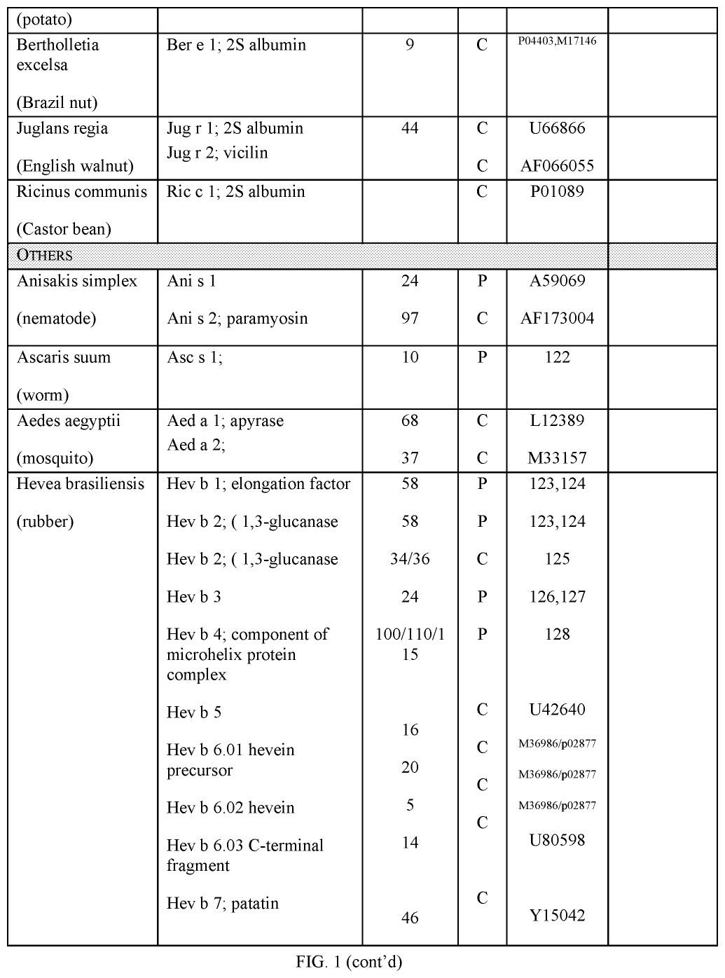

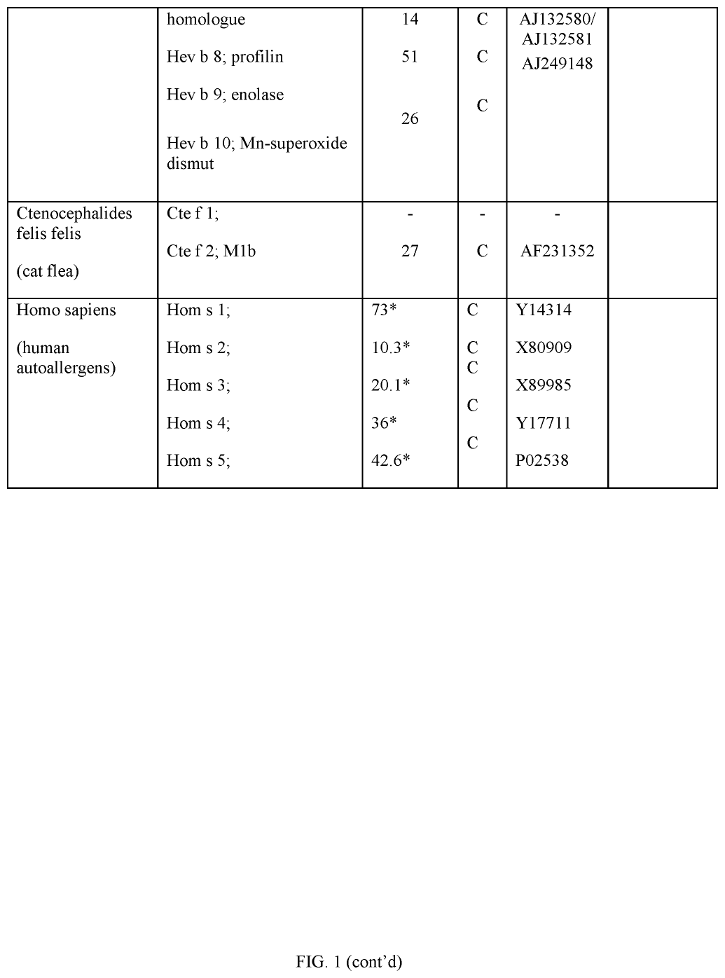

In some embodiments, antigens and/or antigenic extracts comprise or consist of at least one allergic antigen. In an effort to better exemplify some embodiments, an exemplary, non-limiting list of antigens and/or antigenic extracts (such as one or more allergens and/or allergenic extracts, including as found in a list of exemplary allergen sources provided in FIG. 1) that may be used in some embodiments include, but are not limited to, Acarus siro (mite) fatty acid-binding protein (Aca s 13); Actinidia chinensis (kiwi) cysteine protease (Act c 1); Aedes aegyptii (mosquito) antigen (Aed a 2); Aedes aegyptii (mosquito) antigen (Aed a 2); Aedes aegyptii (mosquito) apyrase (Aed a 1); Aedes aegyptii (mosquito) apyrase (Aed a 1); Alnus glutinosa (alder) antigen (Aln g 1); Alternaria alternata (fungus) acid. ribosomal protein P1 (Alt a 12); Alternaria alternata (fungus) aldehyde dehydrogenase (Alt a 10); Alternaria alternata (fungus) antigen (Alt a 1); Alternaria alternata (fungus) antigen (Alt a 2); Alternaria alternata (fungus) enloase (Alt a 11); Alternaria alternata (fungus) heat shock protein (Alt a 3); Alternaria alternata (fungus) ribosomal protein (Alt a 6); Alternaria alternata (fungus) YCP4 protein (Alt a 7); Ambrosia artemisiifolia (short ragweed) antigen E (Amb a 1); Ambrosia artemisiifolia (short ragweed) antigen K (Amb a 2); Ambrosia artemisiifolia (short ragweed) Ra3 antigen (Amb a 3); Ambrosia artemisiifolia (short ragweed) Ra5 antigen (Amb a 5); Ambrosia artemisiifolia (short ragweed) Ra6 antigen (Amb a 6); Ambrosia artemisiifolia (short ragweed) Ra7 antigen (Amb a 7); Ambrosia trifida (giant ragweed) Ra5G antigen (Amb t 5); Anisakis simplex (nematode) antigen (Ani s 1); Anisakis simplex (nematode) paramyosin (Ani s 2); Apis mellifera (honey bee) antigen (Api m 6); Apis mellfera (honey bee) hyaluronidase (Api m 2); Apis mellfera (honey bee) melittin (Api m 4); Apis mellfera (honey bee) phospholipase A2 (Api m 1); Apium graveolens (celery) antigen (Api g 5); Apium graveolens (celery) Bet v 1 homologue (Api g 1); Apium graveolens (celery) profilin (Api g 4); Arachis hypogaea (peanut) (conglutin Ara h 2); Arachis hypogaea (peanut) (profilin Ar a h 5); Arachis hypogaea (peanut) conglutin homologue (Ara h 6); Arachis hypogaea (peanut) conglutin homologue (Ara h 7); Arachis hypogaea (peanut) glycinin (Ara h 3); Arachis hypogaea (peanut) glycinin (Ara h 4); Arachis hypogaea (peanut) vicilin (Ara h 1); Artemisia vulgaris (mugwort) antigen (Art v 1); Artemisia vulgaris (mugwort) antigen (Art v 2); Ascaris suum (worm) antigen (Asc s 1); Aspergillus flavus (fungus) alkaline serine proteinase (Asp fl 13); Aspergillus Fumigatus (fungus) alkaline serine proteinase (Asp f 13); Aspergillus Fumigatus (fungus) antigen (Asp f 1); Aspergillus Fumigatus (fungus) antigen (Asp f 15); Aspergillus Fumigatus (fungus) antigen (Asp f 16); Aspergillus Fumigatus (fungus) antigen (Asp f 17); Aspergillus Fumigatus (fungus) antigen (Asp f 2); Aspergillus Fumigatus (fungus) antigen (Asp f 4); Aspergillus Fumigatus (fungus) antigen (Asp f 7); Aspergillus Fumigatus (fungus) antigen (Asp f 9); Aspergillus Fumigatus (fungus) aspartis protease (Asp f 10); Aspergillus Fumigatus (fungus) heat shock protein P70 (Asp f 12); Aspergillus Fumigatus (fungus) metalloprotease (Asp f 5); Aspergillus Fumigatus (fungus) Mn superoxide dismutase (Asp f 6); Aspergillus Fumigatus (fungus) peptidyl-prolyl isomerase (Asp f 11); Aspergillus Fumigatus (fungus) peroxisomal protein (Asp f 3); Aspergillus Fumigatus (fungus) ribosomal protein P2 (Asp f 8); Aspergillus Fumigatus (fungus) vacuolar serine (Asp f 18); Aspergillus niger (fungus) antigen (Asp n 18); Aspergillus niger (fungus) beta-xylosidase (Asp n 14); Aspergillus niger (fungus) vacuolar serine proteinase; Aspergillus oryzae (fungus) alkaline serine proteinase (Asp o 13); Aspergillus oryzae (fungus) TAKA-amylase A (Asp o 2); Bertholletia excelsa (Brazil nut) 2S albumin (Ber e 1); Betula verrucosa (birch) antigen (Bet v 1); Betula verrucosa (birch) antigen (Bet v 3); Betula verrucosa (birch) antigen (Bet v 4); Betula verrucosa (birch) cyclophilin (Bet v 7); Betula verrucosa (birch) isoflavone reductase homologue (Bet v 5); Betula verrucosa (birch) profilin (Bet v 2); Blattella germanica (German cockroach) aspartic protease (Bla g 2); Blattella germanica (German cockroach) Bd90k (Bla g 1); Blattella germanica (German cockroach) calycin (Bla g 4); Blattella germanica (German cockroach) glutathione transferase (Bla g 5); Blattella germanica (German cockroach) troponin C (Bla g 6); Blomia tropicalis (mite) antigen (Blo t 5); Blomia tropicalis (mite) Btlla antigen (Blo t 12); Blomia tropicalis (mite) Bt6 fatty acid-binding protein (Blo t); Bombus pennsylvanicus (bumble bee) phospholipase (Bom p 1); Bombus pennsylvanicus (bumble bee) protease (Bom p 4); Bos domesticus (cow) Ag3, lipocalin (Bos d 2); Bos domesticus (cow) alpha-lactalbumin (Bos d 4); Bos domesticus (cow) beta-lactalbumin (Bos d 5); Bos domesticus (cow) casein (Bos d 8); Bos domesticus (cow) immunoglobulin (Bos d 7); Bos domesticus (cow) serum albumin (Bos d 6); Brassica juncea (oriental mustard) 2S albumin (Bra j 1); Brassica rapa (turnip) prohevein-like protein (Bar r 2); Candida albicans (fungus) antigen (Cand a 1); Candida boidinii (fungus) antigen (Cand b 2); Canis familiaris (dog) albumin (Can f ?); Canis familiaris (dog) antigen (Can f 1); Canis familiaris (dog) antigen (Can f 2); Carpinus betulus (hornbeam) antigen (Car b 1); Castanea sativa (chestnut) Bet v 1 homologue (Cas s 1); Castanea sativa (chestnut) chitinase (Cas s 5); Chironomus thummi thummi (midge) component I (Chi t 2.0101); Chironomus thummi thummi (midge) component IA (Chi t 2.0102); Chironomus thummi thummi (midge) component II-beta (Chi t 3); Chironomus thummi thummi (midge) component III (Chi t 1.01); Chironomus thummi thummi (midge) component IIIA (Chi t 4); Chironomus thummi thummi (midge) component IV (Chi t 1.02); Chironomus thummi thummi (midge) component IX (Chi t 6.02); Chironomus thummi thummi (midge) component VI (Chi t 5); Chironomus thummi thummi (midge) component VIIA (Chi t 6.01); Chironomus thummi thummi (midge) component VIIB (Chi t 7); Chironomus thummi thummi (midge) component VIII (Chi t 8); Chironomus thummi thummi (midge) component X (Chi t 9); Chironomus thummi thummi (midge) hemoglobin (Chi t 1-9); Cladosporium herbarum (fungus) acid. ribosomal protein P1 (Cla h 12); Cladosporium herbarum (fungus) aldehyde dehydrogenase (Cla h 3); Cladosporium herbarum (fungus) antigen (Cla h 1); Cladosporium herbarum (fungus) antigen (Cla h 2); Cladosporium herbarum (fungus) enolase (Cla h 6); Cladosporium herbarum (fungus) ribosomal protein); Cladosporium herbarum (fungus) YCP4 protein (Cla h 5); Coprinus comatus (shaggy cap) antigen (Cop c 1); Coprinus comatus (shaggy cap) antigen (Cop c 2); Coprinus comatus (shaggy cap) antigen (Cop c 3); Coprinus comatus (shaggy cap) antigen (Cop c 5); Coprinus comatus (shaggy cap) antigen (Cop c 7); Corylus avellana (hazel) antigen (Cor a 1); Corylus avellana (hazelnut) Bet v 1 homologue (Cor a 1.0401); Cryptomeria japonica (sugi) antigen (Cry j 1); Cryptomeria japonica (sugi) antigen (Cry j 2); Ctenocephalides felis felis (cat flea) antigen (Cte f 1); Cynodon dactylon (Bermuda grass) antigen (Cyn d 1); Cynodon dactylon (Bermuda grass) antigen (Cyn d 7); Cynodon dactylon (Bermuda grass) profilin (Cyn d 12); Dactylis glomerata (orchard grass) AgDgl antigen (Dac g 1); Dactylis glomerata (orchard grass) antigen (Dac g 2); Dactylis glomerata (orchard grass) antigen (Dac g 3); Dactylis glomerata (orchard grass) antigen (Dac g 5); Dermatophagoides farinae (mite) antigen (Der f 1); Dermatophagoides farinae (mite) antigen (Der f 2); Dermatophagoides farinae (mite) antigen (Der f 3); Dermatophagoides farinae (mite) Mag 3, apolipophorin (Der f 14); Dermatophagoides farinae (mite) paramyosin (Der f 11); Dermatophagoides farinae (mite) tropomyosin (Der f 10); Dermatophagoides microceras (mite) antigen (Der m 1); Dermatophagoides pteronyssinus (mite) amylase (Der p 4); Dermatophagoides pteronyssinus (mite) antigen (Der p 2); Dermatophagoides pteronyssinus (mite) antigen (Der p 5); Dermatophagoides pteronyssinus (mite) antigen (Der p 7); Dermatophagoides pteronyssinus (mite) antigen P1 (Der p 1); Dermatophagoides pteronyssinus (mite) apolipophorin like p (Der p 14); Dermatophagoides pteronyssinus (mite) chymotrypsin (Der p 6); Dermatophagoides pteronyssinus (mite) collagenolytic serine prot. (Der p 9); Dermatophagoides pteronyssinus (mite) glutathione transferase (Der p 8); Dermatophagoides pteronyssinus (mite) tropomyosin (Der p 10); Dermatophagoides pteronyssinus (mite) trypsin (Der p 3); Dolichovespula arenaria (yellow hornet) antigen 5 (Dol a 5); Dolichovespula maculata (white face hornet) antigen 5 (Dol m 5); Dolichovespula maculata (white face hornet) phospholipase (Dol m 1); Dolichovespula maculate (white face hornet) hyaluronidase (Dol m 2); Equus caballus (horse) lipocalin (Equ c 1); Equus caballus (horse) lipocalin (Equ c 2); Euroglyphus maynei (mite) apolipophorin (Eur m 14); Felis domesticus (cat) cat-1 antigen (Fel d 1); Fraxinus excelsior (ash) antigen (Fra e 1); Gadus callarias (cod) allergen M (Gad c 1); Gallus domesticus (chicken) conalbumin; A22 (Gal d 3); Gallus domesticus (chicken) lysozyme (Gal d 4); Gallus domesticus (chicken) ovalbumin (Gal d 2); Gallus domesticus (chicken) ovomucoid (Gal d 1); Gallus domesticus (chicken) serum albumin (Gal d 5); Glycine max (soybean) antigen (Gly m 2); Glycine max (soybean) HPS (Gly m 1.0101); Glycine max (soybean) HPS (Gly m 1.0102); Glycine max (soybean) profilin (Gly m 3); Haliotis midae (abalone) antigen (Hal m 1); Helianthus annuus (sunflower) antigen (Hel a 1); Helianthus annuus (sunflower) profilin (Hel a 2); Hevea brasiliensis (rubber) 1,3-glucanase (Hev b 2); Hevea brasiliensis (rubber) antigen (Hev b 3); Hevea brasiliensis (rubber) antigen (Hev b 5); Hevea brasiliensis (rubber) component of microhelix protein complex (Hev b 4); Hevea brasiliensis (rubber) C-terminal fragment antigen (Hev b 6.03); Hevea brasiliensis (rubber) elongation factor (Hev b 1); Hevea brasiliensis (rubber) enolase (Hev b 9); Hevea brasiliensis (rubber) hevein (Hev b 6.02); Hevea brasiliensis (rubber) hevein precursor (Hev b 6.01); Hevea brasiliensis (rubber) Mn-superoxide dismut (Hev b 10); Hevea brasiliensis (rubber) patatin homologue (Hev b 7); Hevea brasiliensis (rubber) profilin (Hev b 8); Holcus lanatus (velvet grass) antigen (Hol 1 1); Homo sapiens (human autoallergen) antigen (Hom s 1); Homo sapiens (human autoallergen) antigen (Hom s 2); Homo sapiens (human autoallergen) antigen (Hom s 3); Homo sapiens (human autoallergen) antigen (Hom s 4); Homo sapiens (human autoallergen) antigen (Hom s 5); Hordeum vulgare (barley) BMAI-1 (Hor v 1); Juglans regia (English walnut) 2S albumin (Jug r 1); Juglans regia (English walnut) vicilin (Jug r 2); Juniperus ashei (mountain cedar) antigen (Jun a 1); Juniperus ashei (mountain cedar) antigen (Jun a 3); Juniperus oxycedrus (prickly juniper) calmodulin-like antigen (Jun o 2); Juniperus sabinoides (mountain cedar) antigen (Jun s 1); Juniperus virginiana (eastern red cedar) antigen (Jun v 1); Lepidoglyphus destructor (storage mite) antigen (Lep d 2.0101); Lepidoglyphus destructor (storage mite) antigen (Lep d 2.0102); Ligustrum vulgare (privet) antigen (Lig v 1); Lolium perenne (rye grass) antigen (Lol p Ib); Lolium perenne (rye grass) group I antigen (Lol p 1); Lolium perenne (rye grass) group II antigen (Lol p 2); Lolium perenne (rye grass) group III antigen (Lol p 3); Lolium perenne (rye grass) group IX antigen (Lol p 5); Lolium perenne (rye grass) trypsin (Lol p 11); Malassezia furfur (fungus) antigen (Mal f 1); Malassezia furfur (fungus) antigen (Mal f 4); Malassezia furfur (fungus) antigen (Mal f 5); Malassezia furfur (fungus) cyclophilin homologue (Mal f 6); Malassezia furfur (fungus) MF1 peroxisomal membrane protein (Mal f 2); Malassezia furfur (fungus) MF2 peroxisomal membrane protein (Mal f 3); Malus domestica (apple) Bet v 1 homologue (Mal d 1); Malus domestica (apple) lipid transfer protein (Mal d 3); Mercurialis annua (annual mercury) profilin (Mer a 1); Metapenaeus ensis (shrimp) tropomyosin (Met e 1); Mus musculus (mouse) MUP antigen (Mus m 1); Myrmecia pilosula (Australian jumper ant) antigen (Myr p 1); Myrmecia pilosula (Australian jumper ant) antigen (Myr p 2); Olea europea (olive) antigen (Ole e 1); Olea europea (olive) antigen (Ole e 3); Olea europea (olive) antigen (Ole e 4); Olea europea (olive) antigen (Ole e 6); Olea europea (olive) profilin (Ole e 2); Olea europea (olive) superoxide dismutase (Ole e 5); Oryza sativa (rice) antigen (Ory s 1); Penaeus aztecus (shrimp) tropomyosin (Pen a 1); Penaeus indicus (shrimp) tropomyosin (Pen i 1); Penicillium brevicompactum (fungus) alkaline serine proteinase (Pen b 13); Penicillium citrinum (fungus) alkaline serine proteinase (Pen c 13); Penicillium citrinum (fungus) heat shock protein P70 (Pen c 1); Penicillium citrinum (fungus) peroxisomal membrane protein (Pen c 3); Penicillium notatum (fungus) alkaline serine proteinase (Pen n 13); Penicillium notatum (fungus) N-acetyl glucosaminidase (Pen n 1); Penicillium notatum (fungus) vacuolar serine proteinase (Pen n 18); Penicillium oxalicum (fungus) vacuolar serine proteinase (Pen o 18); Periplaneta americana (American cockroach) Cr—PI (Per a 3); Periplaneta americana (American cockroach) Cr—PII (Per a 1); Periplaneta americana (American cockroach) tropomyosin (Per a 7); Persea americana (avocado) endochitinase (Pers a 1); Phalaris aquatica (canary grass) antigen (Pha a 1); Phleum pratense (timothy grass) antigen (Phl p 1); Phleum pratense (timothy grass) antigen (Phl p 2); Phleum pratense (timothy grass) antigen (Phl p 4); Phleum pratense (timothy grass) antigen (Phl p 6); Phleum pratense (timothy grass) antigen Ag 25 (Phl p 5); Phleum pratense (timothy grass) polygalacturonase (Phl p 13); Phleum pratense (timothy grass) profilin (Phl p 12); Poa pratensis (Kentucky blue grass) antigen (Poa p 5); Poa pratensis (Kentucky blue grass) group I antigen (Poa p 1); Polistes annularies (wasp) antigen 5 (Pol a 5); Polistes annularies (wasp) hyaluronidase (Pol a 2); Polistes annularies (wasp) phospholipase A1 (Pol a 1); Polistes dominulus (Mediterranean paper wasp) antigen (Pol d 1); Polistes dominulus (Mediterranean paper wasp) antigen (Pol d 5); Polistes dominulus (Mediterranean paper wasp) serine protease (Pol d 4); Polistes exclamans (wasp) antigen 5 (Pol e 5); Polistes exclamans (wasp) phospholipase A1 (Pol e 1); Polistes fuscatus (wasp) antigen 5 (Pol f 5); Polistes metricus (wasp) antigen 5 (Pol m 5); Prunus armeniaca (apricot) Bet v 1 homologue (Pru ar 1); Prunus armeniaca (apricot) lipid transfer protein (Pru ar 3); Prunus avium (sweet cherry) Bet v 1 homologue (Pru av 1); Prunus avium (sweet cherry) profilin (Pru av 4); Prunus avium (sweet cherry) thaumatin homologue (Pru av 2); Prunus persica (peach) lipid transfer protein (Pru p 3); Psilocybe cubensis (fungus) antigen (Psi c 1); Psilocybe cubensis (fungus) cyclophilin (Psi c 2); Pyrus communis (pear) Bet v 1 homologue (Pyr c 1); Pyrus communis (pear) isoflavone reductase homologue (Pyr c 5); Pyrus communis (pear) profilin (Pyr c 4); Quercus alba (white oak) antigen (Que a 1); Rattus norvegius (rat) antigen (Rat n 1); Ricinus communis (castor bean) 2S albumin (Ric c 1); Salmo salar (Atlantic salmon) parvalbumin (Sal s 1); Sinapis alba (yellow mustard) 2S albumin (Sin a 1); Solanum tuberosum (potato) patatin (Sol t 1); Solenopsis geminata (tropical fire ant) antigen (Sol g 2); Solenopsis geminata (tropical fire ant) antigen (Sol g 4); Solenopsis invicta (fire ant) antigen (Sol i 2); Solenopsis invicta (fire ant) antigen (Sol i 3); Solenopsis invicta (fire ant) antigen (Sol i 4); Solenopsis saevissima (Brazilian fire ant) antigen (Sol s 2); Sorghum halepense (Johnson grass) antigen (Sor h 1); Syringa vulgaris (lilac) antigen (Syr v 1); Todarodes pacificus (squid) tropomyosin (Tod p 1); Trichophyton rubrum (fungus) antigen (Tri r 2); Trichophyton rubrum (fungus) serine protease (Tri r 4); Trichophyton tonsurans (fungus) antigen (Tri t 1); Trichophyton tonsurans (fungus) serine protease (Tri t 4); Vespa crabo (European hornet) antigen 5 (Vesp c 5.0101); Vespa crabo (European hornet) antigen 5 (Vesp c 5.0102); Vespa crabo (European hornet) phospholipase (Vesp c 1); Vespa mandarina (giant Asian hornet) antigen (Vesp m 1.01); Vespa mandarina (giant Asian hornet) antigen (Vesp m 1.02); Vespa mandarina (giant Asian hornet) antigen (Vesp m 5); Vespula flavopilosa (yellowjacket) antigen 5 (Ves f 5); Vespula germanica (yellowjacket) antigen 5 (Ves g 5); Vespula maculifrons (yellowjacket) antigen 5 (Ves m 5); Vespula maculifrons (yellowjacket) hyaluronidase (Ves m 2); Vespula maculifrons (yellowjacket) phospholipase A1 (Ves m 1); Vespula pennsylvanica (yellowjacket) (antigen 5Ves p 5); Vespula squamosa (yellowjacket) antigen 5 (Ves s 5); Vespula vidua (wasp) antigen (Ves vi 5); Vespula vulgaris (yellowjacket) antigen 5 (Ves v 5); Vespula vulgaris (yellowjacket) hyaluronidase (Ves v 2); Vespula vulgaris (yellowjacket) phospholipase A1 (Ves v 1); Zea mays (maize, corn) lipid transfer protein (Zea m 14); and/or combinations thereof.

In some embodiments, provided compositions and/or methods may include one or more allergens listed in Table 1. In some embodiments, provided compositions and/or methods do not include and or do not utilize allergens or extracts that are substantially pure. In some embodiment, the allergens or extracts are crude. Exemplary crude extracts include, but are not limited to, to extracts derived from the Allergen Source listed in FIG. 1.

In some embodiments, provided compositions may include cancer/tumor antigens. In some embodiments, antigens may be or comprise tumor antigens, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAAO205, Mart2, Mum-1, 2, and 3, neo-PAP, myosin class I, OS-9, pmlRARa fusion protein, PTPRK, K-ras, N-ras, Triosephosphate isomeras, Bage-1, Gage 3,4,5,6,7, GnTV, Herv-K-mel, Lage-1, MageA1, 2,3,4,6,10,12, Mage-C2, NA-88, NY-Eso-1/Lage-2, SP17, SSX-2, and TRP2-Int2, MelanA (MART-I), gplOO (Pmell7), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-25 23H1, PSA, TAG-72-4, CA 19-9, CA 72-4, CAM 17.1, NuMa, K-ras, pCatenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-30 Ag, MOV18, NB\70K, NY—CO-1, RCAS1, SDCCAG16, TA-90 (Mac-2 binding protein\cyclophilin C-associated protein), TAAL6, TAG72, TLP, and TPS.

In some embodiments, cancer antigens are provided in crude form such as a cellular lysate or cellular fraction. Exemplary cellular lysates and/or cellular lysate fractions include, but are not limited to, cancer cells from acute lymphoblastic leukemia (ALL); adrenocortical carcinoma; AIDS-related cancers including AIDS-related lymphoma; anal cancer; appendix cancer; astrocytomas; basal cell carcinoma; bile duct cancer; bladder cancer; bone cancer (e.g., osteosarcoma and malignant fibrous histiocytoma); brainstem glioma; brain cancer; brain tumors; breast cancer; bronchial adenomas/carcinoids; Burkitt lymphoma; carcinoid tumors (e.g., childhood and gastrointestinal tumors); carcinoma (including carcinoma of unknown primary (CUP) whose origin or developmental lineage is unknown but that possess specific molecular, cellular, and histological characteristics of epithelial cells); central nervous system lymphoma; cerebellar astrocytoma; malignant glioma; cervical cancer; childhood cancers; chronic lymphocytic leukemia; chronic myelogenous leukemia; chronic myeloproliferative disorders; colon Cancer; cutaneous T-cell lymphoma; desmoplastic small round cell tumor; endometrial cancer; ependymoma; esophageal cancer; Ewing's sarcoma in the Ewing family of tumors; extracranial germ cell tumor; extragonadal germ cell tumor; ovarian germ cell tumor; extrahepatic bile duct cancer; eye cancer; intraocular melanoma; retinoblastoma; gallbladder cancer; gastric cancer; gastrointestinal carcinoid tumor; gastrointestinal stromal tumor (GIST); gestational trophoblastic tumor; gastric carcinoid; hairy cell leukemia; head and neck cancer; heart cancer; hepatocellular (liver) cancer; Hodgkin lymphoma; hypopharyngeal cancer; hypothalamic and visual pathway glioma; intraocular Melanoma; Islet Cell Carcinoma (Endocrine Pancreas); kaposi sarcoma; soft tissue sarcoma; uterine sarcoma; kidney cancer (renal cell carcinoma); laryngeal cancer; leukemias (including acute lymphoblastic or acute lymphocytic leukemia, acute myeloid or acute myelogenous leukemia, chronic lymphocytic or chronic lymphocytic leukemia, chronic myelogenous or chronic myeloid leukemia); Lip and Oral Cavity Cancer; liposarcoma; liver cancer; lung cancer (including non-small cell and small cell); lymphomas (e.g., AIDS-related, Burkitt, cutaneous T-Cell, Hodgkin, non-Hodgkin, Primary Central Nervous System); macroglobulinemia; medulloblastoma; melanoma; Merkel Cell Carcinoma; mesothelioma (e.g., adult malignant mesothelioma, childhood mesothelioma); metastatic squamous neck cancer; mouth cancer; Multiple Endocrine Neoplasia Syndrome; Multiple Myeloma; Mycosis Fungoides; Myelodysplastic Syndromes; Myelodysplastic/Myeloproliferative Diseases; Myelogenous Leukemia; Myeloid Leukemia; (e.g., Adult Acute; nasal cavity and paranasal sinus cancer; nasopharyngeal carcinoma; neuroblastoma; oral cancer; oropharyngeal cancer; ovarian cancer; ovarian epithelial cancer (Surface epithelial-stromal tumor); ovarian germ cell tumor; ovarian low malignant potential tumor; pancreatic cancer; paranasal sinus and nasal cavity cancer; parathyroid cancer; penile cancer; pharyngeal cancer; pheochromocytoma; pineal astrocytoma; pineal germinoma; pineoblastoma and supratentorial primitive neuroectodermal tumors; pituitary adenoma; pleuropulmonary blastoma; prostate cancer; rectal cancer; renal pelvis and ureter and transitional cell cancer; rhabdomyosarcoma; Sezary syndrome; skin cancer (including melanoma and nonmelanoma); skin carcinoma; small intestine cancer; squamous cell carcinoma; stomach cancer; testicular cancer; throat cancer; thymoma and thymic carcinoma; thyroid cancer; urethral cancer; endometrial uterine cancer; vaginal cancer; vulvar cancer; and/or combinations thereof.

In some embodiments, provided compositions include one or more alloantigens. As described herein, an alloantigen refers to an antigen associated with allorecognition and/or graft rejection (e.g., an antigen against which a rejection immune response is directed). Alloantigens are generally polypeptides expressed by an individual that are genetically different from another individual of the same species. The term “alloantigen polypeptide” refers to a polypeptide whose amino acid sequence includes at least one characteristic sequence of an alloantigen. A wide variety of alloantigen sequences are known in the art.

In some embodiments, an alloantigen for use in accordance with the present disclosure is a major histocompatibility complex (MHC) polypeptide. In some embodiments, an alloantigen for use in accordance with the present disclosure is a Class I MIIC polypeptide. In some embodiments, an alloantigen for use in accordance with the present disclosure is a Class II MIIC polypeptide. In some embodiments, an alloantigen for use in accordance with the present disclosure contains part of or all of an extracellular domain of an MIIC polypeptide. In some embodiments, an alloantigen for use in accordance with the present disclosure is a minor histocompatibility complex polypeptide. In some embodiments, an alloantigen for use in accordance with the present disclosure is a costimulatory entity (e.g., CD28, CD80, and CD86, among others). In some embodiments, an alloantigen for use in accordance with the present disclosure is a non-MHC protein produced by or present in graft tissue and not produced by or present in a host. One of ordinary skill in the art will recognize that alloantigens described herein are exemplary. Any polypeptide that is associated with an allorecognition and/or graft rejection can be classified as an alloantigen.

It will be appreciated that alloantigen polypeptides may have a complete sequence, or alternatively may be polypeptides that represent functional fragments (i.e., fragments retaining at least one activity and/or one characteristic sequence or portion) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another alloantigen polypeptide of the same class, is encompassed within the relevant term “alloantigen polypeptide” as used herein.

### Adjuvant Agents

Adjuvants/adjuvant agents, have historically been used to modify effects of other agents and/or compositions and/or components. Without being bound by a particular theory, an exemplary use of an adjuvant is the addition of an adjuvant to a vaccine, which vaccine is intended to produce immunity against a particular pathogen. Co-administration of an adjuvant (usually by way of combining an adjuvant with the vaccine solution/cocktail) is used to achieve a more robust immune response (e.g. higher titer of antibody to the pathogen against which a subject is being vaccinated).

In accordance with various embodiments, provided compositions may include any of a variety of adjuvant agents (e.g., crude adjuvant agents). In some embodiments, an adjuvant agent may be or comprise a crude adjuvant agent. In some embodiments, provided compositions include a single adjuvant agent (e.g. crude adjuvant agent). In some embodiments, provided compositions include at least 2 adjuvant agents (e.g., 3, 4, 5, 6, 7, 8, 9, 10 or more). In some embodiments, the at least one adjuvant agent includes at least one crude adjuvant agent. In some embodiments, the at least one crude adjuvant agent is or comprises hydrophilic cellular components. In some embodiments, the at least one crude adjuvant agent is or comprises hydrophobic cellular components. In some embodiments, the at least one crude adjuvant agent comprises at least one hydrophobic cellular component and at least one hydrophilic cellular component. In some embodiments, provided methods and/or compositions do not include and/or utilize substantially pure adjuvant agents.

In some embodiments, the at least one crude adjuvant agent is or comprises one more cellular membranes and/or cellular membrane components. In some embodiments, the cellular membrane components do not include transitory vesicles. In some embodiments, the cellular membrane components do not include outer membrane vesicles. In some embodiments, a cellular membrane and/or cellular membrane component is or comprises at least one of a microbial cellular membrane and/or cellular membrane component. In some embodiments, the microbial cellular membrane is or comprises a bacterial cellular membrane. In some embodiments, the bacterial cellular membrane is or comprises an Escherichia coli cellular membrane.

Any of a variety of adjuvant agents may be used in accordance with various embodiments. As used herein, the term “adjuvant agent” refers to a substance that is capable of altering an immune response to an antigen (e.g., a Th2-associated antigen), to enhance or alter the response that would normally occur in a subject in response to that antigen. In some embodiments, for example, administration of a Th2-associated antigen with an adjuvant agent alters a subject's immune response to that antigen such that the primary response to the Th2-associated antigen is not primarily a Th2 response. According to various embodiments, an adjuvant agent is a crude adjuvant agent (i.e., not a pure single molecular entity).

In some embodiments, provided methods and compositions comprise at least one Th2-associated antigen and at least one crude adjuvant agent which are present in a ratio of approximately 1:1 by weight. In some embodiments, provided methods and compositions comprise at least one antigen (e.g., Th2-associated antigen) and at least one crude adjuvant agent which are present in a ratio of greater than approximately 1:1 by weight. In some embodiments, provided methods and compositions comprise at least one antigen (e.g., Th2-associated antigen) and at least one crude adjuvant agent which are present in a ratio of less than approximately 1:1 by weight.

Without wishing to be held to a particular theory, it is possible that some embodiments may mimic one or more characteristics or features of microbial (e.g., bacterial) cells. In some embodiments, adjuvant agents (e.g., crude adjuvant agents) may be provided from one or more bacterial sources, including bacterial cellular lysates and/or cellular lysate fractions. In some embodiments, bacterial cellular lysate fractions are or comprise entities known as pathogen-associated molecular patterns (“PAMPs”). In some embodiments, one or more of a hydrophobic bacterial cellular lysate fraction and/or hydrophilic bacterial cellular lysate fraction include one or more PAMPs as a hydrophilic cellular component and/or hydrophobic cellular component.

PAMPs are generally understood to be entities associated with bacterial cells that are recognized by cells of the innate immune system. In some embodiments, PAMPs are recognized by Toll-like receptors (TLRs) and other pattern recognition receptors (PRRs) in both plants and animals. In some embodiments, PAMPs are recognized by C-type lectin receptors (CLRs). In some embodiments, a CLR is a type I or type II CLR. In some embodiments, PAMPs are or comprise entities associated with the outer surface of a bacterial cell, including, but not limited to, membrane-associated proteins and/or peptides, receptors embedded in bacterial membranes, etc. Exemplary PAMPs include, but are not limited to, bacterial lipopolysaccharide (LPS), bacterial flagellin, lipoteichoic acid from gram positive bacteria, peptidoglycan, double-stranded RNAs (dsRNAs), unmethylated CpG motifs, any of the TLR ligands presented in Table 1, characteristic portions thereof, and/or combinations thereof.

TABLE 1
Exemplary TLRs and TLR Ligands

| TLR | TLR Ligand(s) |
|---|---|
| TLR1 | Multiple triacyl lipopeptides (e.g., |
| | from bacteria and mycobacteria), |
| | such as lipopeptide Pam3Cys-SK4 (“Pam”) |
| TLR2 | Multiple glycolipids, lipopeptides and lipoproteins, |
| | such as lipopeptide Pam3Cys-SK4 (“Pam”) |
| | Lipoteichoic acid |
| | Peptidoglycan |
| | HSP70 |
| | Zymosan |
| | Heat shock proteins, such as Hsp60 |
| TLR3 | Double-stranded RNA |
| | Single-stranded RNA |
| | Poly(I:C) |
| TLR4 | lipopolysaccharide (LPS) |
| | Monophosphoryl lipid A (MPL) |
| | Several heat shock proteins |
| | Fibrinogen |
| | Heparin sulfate fragments |
| | Hyaluronic acid fragments |
| TLR5 | Flagellin |
| TLR6 | Multiple diacyl lipopeptides |
| | Lipoteichoic acid (LTA) |
| | Zymosan |
| TLR7 | Imidazoquinolines (e.g, imiquimod and resiquimod) |
| | Single-stranded RNA, such as GU-rich single-stranded RNA |
| | Loxoribine (a guanosine analog) |
| | Bropirime |
| TLR8 | Imidazoquinolines (e.g, imiquimod and resiquimod) |
| | GU-rich single-stranded RNA |
| | Small synthetic compounds |
| | Single-stranded RNA |
| TLR9 | Unmethylated CpG DNA |
| | Hemazoin crystals |
| | Double-stranded DNA |
| TLR10 | |
| TLR11 | Toxoplasma gondii profilin |
| | Uropathogenic-bacteria-derived protein |

In some embodiments, an adjuvant agent (e.g., crude adjuvant agent) is or comprises a mucosal adjuvant (i.e., an adjuvant capable of eliciting or enhancing an immune response to a mucosally administered antigen). Exemplary mucosal adjuvants include, but are not limited to, TLR4 ligands (e.g., LPS, MPL), cytokines (e.g., IL-1α), c48/80, R848, Pam3CSK4, CpG(ODN1826), lethal factor (LF), and cholera toxin. It will be recognized by those of skill in the art that particular mucosal adjuvants may induce different immune responses. The skilled artisan will understand and be aware of technologies that may be used to select particular adjuvant agent(s) for use in a particular product or products and such variation is specifically contemplated as within the scope of the present disclosure.

Adjuvant Agents—Microbial Cellular Components

As described herein, the present disclosure encompasses the recognition that certain advantages are achieved when hydrophilic and hydrophobic components of microbial cellular preparations, such as lysates that are separated from one another so that a hydrophilic cellular component preparation and/or a hydrophobic cellular component preparation is/are generated, and one or both of such preparations is/are combined with at least one antigen, for example, at least one Th2-associated antigen, to create the compositions of the present disclosure. Accordingly, in some embodiments, an adjuvant agent (e.g., crude adjuvant agent) may be or comprise one or more hydrophilic and/or hydrophobic microbial cellular preparations and/or components.

In some embodiments, one or more of a hydrophilic cellular component preparation and a hydrophobic cellular component preparation may be provided from a microbial cellular lysate. In such embodiments, a hydrophilic cellular component may be referred to as a microbial hydrophilic cellular component and a hydrophobic cellular component may be referred to as a microbial hydrophobic cellular component. Without wishing to be bound by any particular theory, some embodiments of the present disclosure including one or more of a microbial hydrophilic cellular component and/or a microbial hydrophobic cellular component may permit development and/or production of useful immunomodulatory compositions at least in part because they utilize various evolved attributes of microbial cells relating to their ability to modulate or evade certain human or animal immune reactions.

The present disclosure recognizes the source of a problem with various prior art approaches to providing immunomodulatory compositions. Specifically, the present disclosure encompasses the recognition that use of pure components, and in particular pure adjuvant agents, lose certain advantages, including certain immunological effects, achieved by collections of components and/or crude preparations of one or more components, and particularly by collections that mimic or contain absolute and/or relative amounts as are found in such microbial cells. In some embodiments, the present disclosure encompasses the recognition that use of isolated individual microbial components (e.g., particular CpG and/or LPS molecules) may fail to induce as broad or effective an immune response as could be achieved with an extract that includes multiple components and in some embodiments presents a plurality of components in relative amounts that approximate what is found in nature.

In some embodiments, provided methods and compositions comprise at least one microbial extract, e.g., at least one hydrophilic or hydrophobic extract of microbial cells for use in provided compositions. In some embodiments, such microbial extracts may contain a collection of microbial components that share a chemical feature, so that they associate with other included components and not with excluded components during production of the extract. In some embodiments, extracts may contain at least some cellular components at relative levels comparable to those at which they are present in the cells. Those skilled in the art will be aware of a variety of techniques available to determine presence and/or level of particular components, and to compare such determined level(s) with those observed in intact cells. Moreover, those of ordinary skill in the art will readily appreciate reasonable and expected experimental variation and therefore will be able to determine whether components are present in absolute or relative levels or concentrations in an extract that are reasonably comparable to those at which they are present in cells.

In general, microbial extracts are prepared from microbial cell preparations. Microbial cell preparations are prepared by culturing microbial cells for a period of time and under conditions sufficient to achieve cell growth to a desirable level (e.g., optical density, concentration, colony size, total protein, total DNA, and colony forming units). In some embodiments, microbial cell preparations contain intact cells, and optionally are substantially free of lysed cells. In some embodiments, microbial cell preparations contain lysed cells, and optionally are substantially free of intact cells.

In some embodiments, the present disclosure provides hydrophilic microbial extracts, for example extracts prepared by contacting a microbial cell preparation with a hydrophilic solvent so that hydrophilic cellular components partition into solution in the hydrophilic solvent. The hydrophilic solvent can then be separated from non-solubilized components which may, for example, be precipitated, solubilized in a hydrophobic solvent (optionally not miscible with the hydrophilic solvent), or otherwise separable from the hydrophilic solvent. In some embodiments, hydrophilic cellular components that partition into a hydrophilic solvent include, for example, components that are miscible and/or soluble in such solvent.

Separating

In accordance with various embodiments, any of a variety of separation methods may be used to separate hydrophilic cellular components from hydrophobic cellular components. Exemplary suitable methods include solvent extraction, detergent extraction, and phase separation.

Exemplary hydrophilic components that may be found in certain embodiments of hydrophilic cellular extracts include, but are not limited to, cytosol components; carbohydrates including sugars; amphipathic molecules (e.g., glycolipids and/or lipoproteins); salts; soluble proteins (i.e., polar proteins); nucleic acids (e.g., DNA and/or RNA); and/or combinations thereof. In some embodiments, a hydrophilic cellular extract includes sheared DNA or RNA. In some embodiments, a hydrophilic cellular extract includes lipopolysaccharides (LPS). In some embodiments, a hydrophilic cellular extract includes one or more CpGs. In some embodiments, a hydrophilic cellular extract is substantially free of membrane lipids or membrane proteins.

In some embodiments, the present disclosure provides hydrophobic microbial extracts, for example extracts prepared by contacting a microbial cell preparation with a hydrophobic solvent so that hydrophobic cellular components partition into solution in the hydrophobic solvent. The hydrophobic solvent can then be separated from non-solubilized components which may, for example, be precipitated, solubilized in a hydrophilic solvent (optionally not miscible with the hydrophobic solvent), or otherwise separable from the hydrophobic solvent. In some embodiments, hydrophobic cellular components that partition into a hydrophobic solvent include that are miscible and/or soluble in the solvent; in some embodiments, such hydrophobic cellular components include components that are substantially immiscible and/or insoluble in water and/or other aqueous solvents.

Exemplary components that may be found in some embodiments of hydrophobic cellular extracts include, but are not limited to, cell membrane components; certain carbohydrates including certain glycoproteins and/or glycolipids; certain proteins including certain glycoproteins, transmembrane proteins, lipid anchored proteins (i.e., non-polar proteins); lipids including phospholipids, glycolipids, and cholesterols; and/or combinations thereof. In some embodiments, a hydrophobic cellular extract includes lipopolysaccharides (LPS). In some embodiments, a hydrophobic cellular extract is substantially free of components found exclusively in the cytoplasm (e.g., hydrophilic proteins, DNA, and RNA).

In some embodiments, a hydrophilic extract is substantially free of hydrophobic components; in some embodiments, a hydrophobic extract is substantially free of hydrophilic components. However, as will be appreciated by those skilled in the art, separation of cellular components by extract preparation is often not complete. That is, in some embodiments, at least some cellular components may partition substantially equally into hydrophilic and hydrophobic extracts; other components may partition favorably but not exclusively into one or the other of a hydrophilic and a hydrophobic extract. To give but a few examples, in some embodiments, amphiphilic entities such as for example certain membrane spanning proteins, glycolipids and/or lipoproteins, LPS, etc., and combinations thereof.

In accordance with various embodiments, microbial extracts for use in accordance with the present disclosure can be prepared from extracts of any microbial cells, or combinations thereof. In some embodiments, microbial extracts are prepared from bacterial, fungal, archael, and/or protest cells, or combinations thereof.

In some embodiments, microbial extracts can be prepared from bacterial cells including, but not limited to Actinomyces, Aeromonas, Anabaena, Arthrobacter, Bacillus, Bacteroides, Bdellovibrio, Bordetella, Borrelia, Campylobacter, Caulobacter, Chlamydia, Chlorobium, Chromatium, Citrobacter, Clostridium, Corynebacterium, Cytophaga, Deinococcus, Enterobacter, Escherichia, Francisella, Halobacterium, Heliobacter, Haemophilus, Hemophilus influenza type B (HIB), Hyphomicrobium, Klebsiella, Lactococcus, Legionella, Leptspirosis, Listeria, Meningococcus A, B and C Methanobacterium, Micrococcus, Morganella, Myobacterium, Mycoplasma, Myxococcus, Neisseria, Nitrobacter, Oscillatoria, Peptococcus, Phodospirillum, Plesiomonas, Prochloron, Proteus, Providencia, Pseudomonas, Rickettsia, Salmonella, Serratia, Shigella, Spirillum, Spirochaeta, Sporolactobacillu, Staphylococcus, Streptococcus, Streptomyces, Sulfolobus, Thermoplasma, Thiobacillus, and Treponema, Vibrio, Yersinia, and combinations thereof. In some embodiments, microbial extracts can be prepared from E. coli cells.

In some embodiments, microbial extracts can be prepared from yeast cells such as, for example, Brettanomyces anomalus, Brettanomyces bruxellensis, Brettanomyces claussenii, Brettanomyces custersianus, Brettanomyces lambicus, Brettanomyces naardenensis, Brettanomyces nanus, Canida albicans, Candida blankii, Candida slooffi, Dekkera intermedia, Leucosporidium frigidum, Rhodotorula rubra, Saccharomyces cerevisiae, Saccharomyces pastorianus, Saccharomyces telluris, Schizosaccharomyces pombe, Sporidiobolus johnsonii, Sporidiobolus longiusculus, Sporidiobolus metaroseus, Sporidiobolus pararoseus, Sporidiobolus ruineniae, Sporidiobolus salmonicolor, Sporidiobolus veronae, Trichosporon beigei, Trichosporon cutaneum, and combinations thereof. In some embodiments, microbial extracts can be prepared from S. cerevisiae cells.

In some embodiments, microbial extracts can be prepared from one or more microbial cell types that are pathogenic in the organism to which inventive compositions are to be administered. In some embodiments, microbial extracts can be prepared from one or more microbial cell types that naturally colonize subjects to which inventive compositions are to be administered. In some embodiments, microbial extracts can be prepared from microbial cell types that are present in foods consumed by organisms to which inventive compositions are to be administered.

Without wishing to be held to a particular theory, some embodiments may be beneficial and/or desirable in their ability to recreate natural environmental exposure to one or more substances. For example, in some embodiments, the presence of a mix of naturally occurring microbial extract components may replicate an environmental exposure to one or more toxins, infectious agents, antigens and/or allergens.

Associating

Any of a variety of methods of associating one or more adjuvant agents (e.g., cellular preparations, cellular extracts, one or more antigen preparations, and/or one or more other agents) with an antigen (e.g., a Th2-associated antigen) may be used according to various embodiments. Exemplary methods of associating include, but are not limited to: mixing, blending or combining under pressure substantially equal to atmospheric pressure, mixing, blending or combining under pressures elevated above atmospheric pressure, mixing, blending or combining under pressure less than atmospheric pressure (e.g. vacuum), spray-drying, etc. As exemplified herein, any of a variety of manners of association between the at least one antigen and the at least one adjuvant agent may be used, in accordance with various embodiments. For example, in some embodiments, association is or comprises a covalent association. By way of additional example, in some embodiments, association is or comprises a non-covalent association. In some embodiments, association may be or comprise co-localization.

In some embodiments, one or more antigens (e.g., Th2-associated antigens) and adjvant agents (e.g., crude adjuvant agents) are associated covalently. In some embodiments, one or more antigens (e.g., Th2-associated antigens) and adjvant agents (e.g., crude adjuvant agents) are associated non-covalently. In some embodiments, non-covalent association involves incorporation of one or more components into or onto an article. In some embodiments, non-covalent association involves specific binding with an article and/or an element incorporated therein. In some specific embodiments, one or more particular components of an extract, preparation, or agent may be coupled with a ligand that specifically binds with a target in the article. In some embodiments, a ligand-target combination utilized in such an embodiment may be, for example, biotin-avidin, antibody-antigen, GST-glutathione, mannose binding protein-mannose, Protein A-IgG, and/or S-tag.

In some embodiments, provided compositions may include a plurality of compositions that share one or more structural and/or functional characteristics. For example, in some embodiments, provided compositions may comprise a plurality of sets of crude adjuvant agents. Alternatively or additionally, in some embodiments, provided compositions may comprise a plurality of sets each of which is designed to have and/or is characterized by a different half-life (e.g., in a relevant tissue or organ of interest), different components (e.g., in an article, different populations of antigens, etc). In some embodiments, provided compositions and/or methods do not include and/or utilize nanoparticles and/or microparticles.

Pharmaceutical Compositions

In some embodiments, the present disclosure provides pharmaceutical compositions comprising a provided composition together with one or more pharmaceutically acceptable carriers or excipients.

In some embodiments, provided pharmaceutical compositions may be prepared by any appropriate method, for example as known or hereafter developed in the art of pharmacology. In general, such preparatory methods include the step of bringing a provided composition into association with one or more pharmaceutically acceptable excipients, and then, if necessary and/or desirable, shaping and/or packaging the product into an appropriate form for administration, for example as or in a single- or multi-dose unit.

In some embodiments, compositions may be prepared, packaged, and/or sold in bulk, as a single unit dose, and/or as a plurality of single unit doses. As used herein, a “unit dose” is a discrete amount of the pharmaceutical composition comprising a predetermined amount of the provided composition. The amount of the provided composition is generally equal to the dosage of the provided composition which would be administered to a subject and/or a convenient fraction of such a dosage such as, for example, one-half or one-third of such a dosage.

In many embodiments, provided pharmaceutical compositions are specifically formulated for mucosal delivery (e.g., oral, nasal, rectal or sublingual delivery). In some embodiments, provided pharmaceutical compositions do not include and/or utilize nanoparticles and/or microparticles.

In accordance with various embodiments, provided compositions may further include one or more medically appropriate substances (e.g., therapeutic agents). For example, in some embodiments, provided compositions further include at least one pharmaceutically acceptable carrier.

In some embodiments, appropriate excipients for use in provided pharmaceutical compositions may, for example, include one or more pharmaceutically acceptable solvents, dispersion media, granulating media, diluents, or other liquid vehicles, dispersion or suspension aids, surface active agents and/or emulsifiers, isotonic agents, thickening or emulsifying agents, preservatives, solid binders, lubricants, disintegrating agents, binding agents, preservatives, buffering agents and the like, as suited to the particular dosage form desired. Alternatively or additionally, pharmaceutically acceptable excipients such as cocoa butter and/or suppository waxes, coloring agents, coating agents, sweetening, flavoring, and/or perfuming agents can be utilized. Remington's The Science and Practice of Pharmacy, 21^{st} Edition, A. R. Gennaro (Lippincott, Williams & Wilkins, Baltimore, Md., 2005; incorporated herein by reference) discloses various excipients used in formulating pharmaceutical compositions and known techniques for the preparation thereof.

In some embodiments, an appropriate excipient is at least 95%, at least 96%, at least 97%, at least 98%, at least 99%, or 100% pure. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or other International Pharmacopoeia.

In some embodiments, liquid dosage forms (e.g., for oral and/or parenteral administration) include, but are not limited to, emulsions, microemulsions, solutions, suspensions, syrups, and/or elixirs. In addition to provided compositions, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such a CREMOPHOR, alcohols, oils, modified oils, glycols, polysorbates, cyclodextrins, polymers, and/or combinations thereof.

In some embodiments, injectable preparations, for example, sterile aqueous or oleaginous suspensions, may be formulated according to known methods using suitable dispersing agents, wetting agents, and/or suspending agents. Sterile liquid preparations may be, for example, solutions, suspensions, and/or emulsions in nontoxic parenterally acceptable diluents and/or solvents, for example, as a solution in 1,3-butanediol. Among the acceptable vehicles and solvents that may be employed, for example, are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of liquid formulations.

Liquid formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, one or more strategies may be utilized to prolong and/or delay the effect of a provided composition after delivery. In some embodiments, a strategy may be considered to prolong or delay based on a comparison with an appropriate reference such as, for example, a) no treatment, or b) treatment with the current standard of care.

In some embodiments, provided pharmaceutical compositions may be formulated as suppositories, for example for rectal or vaginal delivery. In some embodiments, suppository formulations can be prepared by mixing, utilizing suitable non-irritating excipients such as cocoa butter, polyethylene glycol or a suppository wax which are solid at ambient temperature but liquid at body temperature and therefore melt in the body (e.g., in the rectum or vaginal cavity) and release the provided composition.

In some embodiments, solid dosage forms (e.g., for oral administration) include capsules, tablets, pills, powders, and/or granules. In such solid dosage forms, a provided composition may be mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato starch, tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents. In some embodiments, the dosage form does not include and/or utilize nanoparticles and/or microparticles.

In some embodiments, solid compositions of a similar type may be employed as fillers in soft and/or hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

Exemplary enteric coatings include, but are not limited to, one or more of the following: cellulose acetate phthalate; methyl acrylate-methacrylic acid copolymers; cellulose acetate succinate; hydroxy propyl methyl cellulose phthalate; hydroxy propyl methyl cellulose acetate succinate (hypromellose acetate succinate); HP55; polyvinyl acetate phthalate (PVAP); methyl methacrylate-methacrylic acid copolymers; methacrylic acid copolymers, cellulose acetate (and its succinate and phthalate version); styrol maleic acid co-polymers; polymethacrylic acid/acrylic acid copolymer; hydroxyethyl ethyl cellulose phthalate; hydroxypropyl methyl cellulose acetate succinate; cellulose acetate tetrahydrophtalate; acrylic resin; shellac, and combinations thereof.

In some embodiments, solid dosage forms may optionally comprise opacifying agents and can be of a composition that they release the provided composition(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In some embodiments, the present disclosure provides compositions for topical and/or transdermal delivery, e.g., as a cream, patch, liniment, ointment, oil, foam, spray, lotion, liquid, powder, thickening lotion, or gel. Particular exemplary such formulations may be prepared, for example, as products such as skin softeners, nutritional lotion type emulsions, cleansing lotions, cleansing creams, skin milks, emollient lotions, massage creams, emollient creams, make-up bases, lipsticks, facial packs or facial gels, cleaner formulations such as shampoos, rinses, body cleansers, hair-tonics, or soaps, or dermatological compositions such as lotions, ointments, gels, creams, liniments, patches, deodorants, or sprays. In some embodiments, provided compositions and/or formulations do not include and/or utilize nanoparticles and/or microparticles.

In some embodiments, an adjuvant is provided in the same formulation with provided composition(s) so that adjuvant and provided composition are delivered substantially simultaneously to the individual. In some embodiments, an adjuvant is provided in a separate formulation. Separate adjuvant may be administered prior to, simultaneously with, or subsequent to administration of provided compositions.

In some embodiments, provided compositions are stable for extended periods of time, such as 1 week, 2 weeks, 1 month, 2 months, 6 months, 1 year, 2 years, 3 years, or more. In some embodiments, provided compositions are easily transportable and may even be sent via traditional courier or other package delivery service. Accordingly, some embodiments may be useful in situations of disease outbreak, such as epidemics, or attacks with biological agents (e.g. anthrax, smallpox, viral hemorrhagic fevers, plague, and others) at least in part due to their ability to be stored for long periods of time and transported quickly, easily, and safely. Such attributes may allow for rapid distribution of provided compositions to those in need.

In some embodiments, it may be advantageous to release encapsulated agent, for example, an antigen, at various locations along a subject's gastrointestinal (GI) tract. In some embodiments, it may be advantageous to release encapsulated agent, for example, an antigen, in a subject's mouth as well as one or more locations along the subject's GI tract. Accordingly, in some embodiments, a plurality of provided compositions (e.g., two or more) may be administered to a single subject to facilitate release of encapsulated agent at multiple locations. In some embodiments, each of the plurality of compositions has a different release profile, such as provided by various enteric coatings, for example. In some embodiments, each of the plurality of compositions has a similar release profile. In some embodiments, the plurality of compositions comprises one or more antigens. In some embodiments, each of the plurality of administered compositions comprises a different antigen. In some embodiments, each of the plurality of compositions comprises the same antigen.

In some embodiments, one or more agents may be included that can affect rate and/or extent of release of Th2-associated antigens from provided compositions. In some embodiments, such an agent would affect rate and/or extent of release by leakage or otherwise undesired release (e.g., at a site other than a target site and/or at a time other than a desired time).

### Articles

Any of a variety of articles may be used in accordance with various embodiments. As used herein, the term “article” refers to a particular item which, at the outset is tangible and which retains one or more components upon and/or within itself. In some embodiments, at least one antigen and at least one adjuvant are co-localized with, within, or on one or more articles. In some embodiments, an article co-localizes at least one antigen and at least one adjuvant such that treatment of a subject in need thereof results in improved immunomodulation, relative to immunomodulation achieved via an appropriate reference treatment. In some embodiments, an appropriate reference treatment is the at least one antigen and at least one adjuvant administered without colocalization. In some embodiments, an appropriate reference is a pure or substantially pure form of the at least one adjuvant or at least one antigen. In some embodiments, the one or more components may or may not be released from an article through, e.g. diffusion, or other means. In some embodiments, the article may be capable of being degraded or otherwise broken down, such as absorption, dissolution, etc. In some embodiments, the present disclosure provides compositions comprising a provided composition together with at least one article. In some embodiments, at least one antigen (e.g., a Th2-associated antigen) and at least one adjuvant agent (e.g., crude adjuvant agent) are retained within the article.

Various embodiments may include association (e.g., partial or total encapsulation) of provided compositions with or in one or more articles. In some embodiments, provided compositions further include at least one article within which at least one antigen and at least one adjuvant agent (e.g., crude adjuvant agent) are retained. In some embodiments, an article comprises one or more compartments. In some embodiments, an article is selected from the group consisting of a patch, capsule, tablet, lyophilized solid, gel, matrix, paste, reservoir, adhesive, liquid, suspension, liquid contained within one or more lumenal units, and combinations thereof. In some embodiments, an antigen and at least one adjuvant agent are in the same compartment. In some embodiments, an antigen and at least one adjuvant agent are in different compartments. In some embodiments, a gel is or comprises a hydrogel. In some embodiments, an article does not include and/or utilize nanoparticles and/or microparticles.

In some embodiments, the article is a single or multi-layer object. In some embodiments, the article comprises one or more compartments. In some embodiments, the compartments contain one or more sub-components. In some embodiments, the components may or may not be associated with one another at any given time while retained within the article. In some embodiments, the components are not or do not include nanoparticles and/or microparticles.

In some embodiments, the article is selected from a group consisting of a: patch, capsule, particle, tablet, gel, matrix, paste, reservoir, adhesive, liquid, suspension, lyophilized solid, liquid contained within one or more lumenal units, and combinations thereof. In some embodiments, a gel is or comprises a hydrogel.

In some embodiments, the present disclosure provides a kit comprising at least one antigen (e.g., Th2-associated antigen), at least one adjuvant agent (e.g., crude adjuvant agent) and at least one article and/or at least one pharmaceutically acceptable excipient.

In some embodiments an article may be a specific item or object upon, within, or among which the composition is placed, mixed, administered, and/or stored.

In some embodiments, an article may be comprised of one or more natural and/or synthetic components.

In some embodiments, the article is used to administer one or more components or one or more provided compositions and/or formulations. In some embodiments, the article is placed on or within and/or otherwise administered to a patient in need thereof.

Routes of Administration

In some embodiments, provided compositions may be formulated for any appropriate route of delivery (e.g. medically appropriate route of delivery). In some embodiments, provided compositions may be formulated for any route of delivery, including, but not limited to, bronchial instillation, and/or inhalation; buccal, enteral, interdermal, intra-arterial (IA), intradermal, intragastric (IG), intramedullary, intramuscular (IM), intranasal, intraperitoneal (IP), intrathecal, intratracheal instillation (by), intravenous (IV), intraventricular, mucosal, nasal spray, and/or aerosol, oral (PO), as an oral spray, rectal (PR), subcutaneous (SQ), sublingual; topical and/or transdermal (e.g., by lotions, creams, liniments, ointments, powders, gels, drops, etc.), transdermal, vaginal, vitreal, and/or through a portal vein catheter; and/or combinations thereof. In some embodiments, the present disclosure provides methods of administration of provided compositions via mucosal administration. In some embodiments, the present disclosure provides methods of administration of provided compositions via oral administration. In some embodiments, the present disclosure provides methods of administration of provided compositions via sublingual administration.

In some embodiments, the administration is topical and/or transdermal. In some embodiments, administration of provided compositions results in immune tolerance to the antigen. In some embodiments, the tolerance is characterized by one or more of: an increased Th1 response and/or a lack of eliciting a Th2 response, wherein the Th2 response causes one or more of skin reactions wherein the skin reactions comprise one or more of hives, flushed skin, pale skin, itchiness of skin, swelling of skin; increase or feeling of increase in body temperature; tightness or feeling of tightness of the throat; constriction of airways; swelling of the tongue; swelling of the throat; difficulty breathing; weak and/or rapid pulse, nausea; vomiting; diarrhea; dizziness; fainting; and sudden or sharp decrease in blood pressure.

In some embodiments, administration of provided compositions results in immune desensitization to the antigen. In some embodiments, the desensitization is characterized by a decreased Th2 response. In some embodiments, the desensitization is characterized by an increased Th1 response.

Methods of Treatment

The present disclosure provides, among other things, methods including the step of administering a composition provided herein to a subject in need thereof. In some embodiments, the subject is suffering from a condition that results in a host immune response. In some embodiments, the subject is suffering from a disease or infection. In some embodiments, the subject is suffering from cancer. In some embodiments, the subject is suffering, has suffered from, or is susceptible to at least one allergic response to at least one antigen.

In some embodiments, the present disclosure provides methods of treating various diseases, disorders and/or conditions. In some embodiments, provided compositions may be administered to a subject for treatment and/or prevention of allergy, infection, cancer, and combinations thereof. Exemplary suitable compositions include those described herein.

Diseases, Disorders, and Conditions Associated with an Undesirable Immune Response

Allergy

The present disclosure provides, among other things, methods and compositions for the treatment and/or prevention of allergy. In some embodiments, provided compositions are useful as vaccines to prevent and/or delay the onset of an allergic reaction. In some embodiments, provided compositions are useful as vaccines to lessen the severity and/or duration of a future allergic reaction. In some embodiments, provided compositions are useful as therapeutics to alleviate and/or arrest an allergic reaction in progress. In some embodiments, the subject in need thereof is suffering from an allergic condition as herein described, including, but not limited to allergic rhinitis, asthma, atopic eczema, anaphylaxis, insect venom, drug allergies, food allergies, and/or combinations thereof.

In some embodiments, provided compositions may be used for treatment and/or prevention of allergies associated with anaphylactic allergens, e.g., food allergens, insect allergens, and rubber allergens (e.g., from latex).

In some embodiments, provided compositions may be used for treatment and/or prevention of allergies associated with food. Food allergies are mediated through the interaction of IgE to specific proteins contained within the food. Examples of common food allergens include proteins from nuts (e.g., from peanut, walnut, almond, pecan, cashew, hazelnut, pistachio, pine nut, brazil nut), dairy products (e.g., from egg, milk), seeds (e.g., from sesame, poppy, mustard), soybean, wheat, and fish (e.g., shrimp, crab, lobster, clams, mussels, oysters, scallops, crayfish).

In some embodiments, provided compositions may be used for treatment and/or prevention of allergies associated with insect allergens. Examples of common insect allergens include, but are not limited to, proteins from insects such as fleas, ticks, ants, cockroaches, and bees.

In some embodiments, allergens elicit a reaction when ingested, inhaled, and/or injected. Allergens can also elicit a reaction based solely on contact with the skin. Latex is a well-known example. Latex products are manufactured from a milky fluid derived from the rubber tree (Hevea brasiliensis) and other processing chemicals. A number of the proteins in latex can cause a range of allergic reactions. Many products contain latex, such as medical supplies and personal protective equipment. Two types of reactions can occur in persons sensitive to latex: local allergic dermatitis and immediate systemic hypersensitivity (or anaphylaxis).

In some embodiments, provided compositions may be used for treatment and/or prevention of allergies associated with local allergic dermatitis. Local allergic dermatitis may develop within a short time after exposure to latex and generally includes symptoms of urticaria or hives. The reaction is thought to be allergic and triggered by direct contact, not inhalation (Sussman et al., 1991, JAMA, 265:2844; incorporated herein by reference). Symptoms of immediate systemic hypersensitivity vary from skin and respiratory problems (e.g., urticaria, hives, rhinoconjunctivitis, swelling of lips, eyelids, and throat, wheezing, and coughing) to anaphylaxis which may progress to hypotension and shock. Such a reaction may be triggered by inhalation or skin exposure to the allergen.

In some embodiments, provided compositions may function to suppress and/or decrease a subject's T_{H}2-type responses and/or enhance and/or increase a subject's T_{H}1-type responses. In some embodiments, provided compositions may function to enhance and/or increase a subject's T_{H}2-type responses and/or suppress and/or decrease a subject's T_{H}1-type responses. In some embodiments, a subject's T_{H}2-type responses are enhanced through targeting of a cell surface receptor for CpG oligonucleotides (e.g. DEC205).

In some embodiments, provided compositions effectively treat and/or prevent all of a subject's allergies falling into a particular class of allergy. In some embodiments, exemplary “classes” of allergies include, but are not limited to, anaphylactic allergies and non-anaphylactic allergies. In some embodiments, exemplary “classes” of allergies include, but are not limited to food allergies, insect allergies, pet dander allergies, pollen allergies, grass allergies, rubber allergies, and so forth. Thus, in some embodiments, provided compositions may be useful for treating all of a subject's food allergies. In some embodiments, exemplary “classes” of allergies include, but are not limited to, particular individual foods which contain multiple allergens. For example, there are at least eleven known peanut allergen proteins. Thus, in some embodiments, a “class” of allergies is “peanut” allergy, and provided compositions may be useful for treating all of a subject's allergies associated with all seven different peanut allergen proteins.

In some embodiments, provided compositions may be useful for treating and/or preventing a single allergy, even though no allergy-specific antigen is included. In some embodiments, provided compositions may be useful for treating and/or preventing multiple different allergies. In some embodiments, provided compositions may be useful for treating and/or preventing substantially all of a subject's allergies. For example, subjects suffering from and/or susceptible to allergy are frequently allergic to more than one allergen, e.g., at least 1, at least 2, at least 3, at least 4, at least 5, at least 6, at least 7, at least 8, at least 9, at least 10, at least 15, at least 20, at least 25, or more different allergens. Thus, in some embodiments, a provided composition may be used for treating and/or preventing at least 1, at least 2, at least 3, at least 4, at least 5, at least 6, at least 7, at least 8, at least 9, at least 10, at least 15, at least 20, at least 25, or more different allergies in a single patient. In some embodiments, a provided composition is administered to a subject suffering from and/or susceptible to multiple different allergies, e.g., at least 1, at least 2, at least 3, at least 4, at least 5, at least 6, at least 7, at least 8, at least 9, at least 10, at least 15, at least 20, at least 25, or more different allergies, such that the subject's symptoms are reduced and/or improved. In some embodiments, a provided composition is administered to a subject suffering from and/or susceptible to multiple different allergies, e.g., at least 1, at least 2, at least 3, at least 4, at least 5, at least 6, at least 7, at least 8, at least 9, at least 10, at least 15, at least 20, at least 25, or more different allergies, such that onset of the subject's symptoms is delayed. In some embodiments, a strategy may be considered to prolong or delay based on a comparison with an appropriate reference such as, for example, a) no treatment, or b) treatment with the current standard of care.

In some embodiments, provided compositions may be used as oral vaccines to treat allergy. One of the major benefits of oral vaccines is the ability to generate both mucosal and systemic immunity. While oral vaccines have been developed previously, but they have been almost entirely directed to prevention of infectious disease, and have met with widely varying levels of success. For example, oral vaccines have been developed for anthrax, cholera, gastoenteritis, infant diarrhea, malaria, measles, and tuberculosis, among others (see Aziz et al., Oral Vaccines: New Needs, New Possibilities, 2007, BioEssays 29.6: 591-604; see also Silin et al., Oral Vaccination: Where are we?, Exp. Opin. Drug Deliv., 2007, 4(4):323-340, both of which are hereby incorporated by reference in their entirety). Part of the reason for such unpredictable results is the complex nature of the gut mucosa. Briefly, the base of the mucosa in the gut is lined by gut- or mucosa-associated lymphoid tissue, with underlying lamina propria that is rich in intraepithelial lymphocytes (sometimes referred to as diffuse lymphoid tissue). The majority of T-cells in the gut mucosa are either αβ or γδ types. Both CD4 and CD8 cells are found in the gut mucosa, which also carries B cells, monocytes/macrophages, dendrocytes and other immune cells. In fact, the gut is known to house ˜90% of the total number of immunocompetent cells in the human body, with circulating lymphocytes only comprising ˜2% of the total lymphocytes (see Silin et al.). Furthermore, the gut is known to accommodate ˜80% of all immunoglobin or Ig-producing cells and releases 2 to 3 times more secretory IgA that the total output of circulating IgG (see Silin et al.). Accordingly, any therapy that is exposed to the gut environment has the potential to engender a wide variety of responses and be affected by any of several immune or other cells.

In order to have an effective oral vaccine to treat allergy, effective presentation of one or more antigens to an antigen presenting cell (APC) is required. While M-cells and Peyer's patches are popular targets of oral therapies, additional targets include, but are not limited to, enterocytes, mesenteric lymph nodes, and intestinal epithelial cells. Each APC may be targeted by various embodiments. Oral immunization is known to generate significant quantities of secretory IgA (sIgA), which is known to play a major role in mucosal defense against pathogens. However, the value of sIgA is questionable when one considers non-mucosal pathogens or conditions. Various embodiments recognize this and do not trigger large amounts of sIgA release, instead substantially generating a Th2 response.

Major known barriers to providing effective oral vaccines include proteolytic degradation of antigens in the gut, tuning of proper release profile in the intestine, and problems delivering enough antigen in a reasonable sized dose. Additionally, the development of oral tolerance to an antigen is thought to be a major point of concern in developing oral vaccines in general. Oral tolerance is a phenomenon where oral antigen exposure can lead to immune tolerance and a suppression of the systemic immune response to subsequent challenges. The development of oral tolerance is not an automatic feature of oral antigen exposure, but rather depends on several factors including, but not limited to, age of subject, MHC restriction, delivery site, nature, size and dose of antigen, degree of antigenic uptake, and processing and frequency of administration of antigen. Oral tolerance is thought to be mediated by several immunological mechanisms including: induction of regulatory T-cells (suppressors) that downregulate specific cytokines including IL-4, IL-10, and TGF-β, functional of clonal deletion of effector cells, and antibody-mediated suppression (see Silin et al.).

In some embodiments, provided compositions are able to present antigen to APCs without inducing oral tolerance. Without wishing to be held to a particular theory, it is possible certain embodiments are able to present larger quantities of antigen to the immune system than traditionally known methods of oral immunization. It is suspected that oral tolerance may manifest, at least in part, due to very small amounts of antigen being presented to APCs (see Silin et al., Overcoming immune tolerance during oral vaccination against actinobacillus pleuropneumoniae, 2002, J Vet. Med. 49:169-175). In some embodiments, provided compositions present antigens to APCs in such a manner as to promote immune tolerance. Without wishing to be held to a particular theory, it may be advantageous to promote immune tolerance in some clinical circumstances, such as in cases of anaphylaxis, autoimmune disease, or certain infectious diseases including, but not limited to, dengue fever and RSV.

Infection

In some embodiments, the subject in need thereof is suffering from an infection caused by, but not limited to viruses, prions, bacteria, viroids, macroparasites, fungi, and/or combinations thereof. In some embodiments, the subject is suffering from a primary infection. In some embodiments, the subject is suffering from a secondary infection. In some embodiments, the subject is suffering from an active symptomatic infection. In some embodiments, the subject is suffering from an active asymptomatic infection (i.e., infection is active, but does not produce noticeable symptoms; e.g. silent or subclinical infection). In some embodiments, the subject is suffering from a latent infection (i.e., inactive or dormant infection).

Exemplary infections that may be treated by some embodiments include, but are not limited to actinomycosis, African sleeping sickness, AIDS, anthrax, hemorrhagic fevers, bacterial pneumonia, candidiasis, cellulitis, Chagas disease, chicken pox, cholera, C. difficile infection, Creutzfeldt-Jakob disease, dengue fever, diphtheria, ebola, enterococcus infection, food poisoning, gangrene, gonorrhea, streptococcal infections, hepatitis A-E, herpes, hookworm, mononucleosis, leishmaniosis, leprosy, Lyme disease, malaria, measles, meningitis, mumps, conjunctivitis, pertussis, rabies, respiratory syncytial virus, rhinovirus, rubella, SARS, scabies, sepsis, shingles, syphilis, tetanus, trichinellosis, tuberculosis, tularemia, viral pneumonia, west nile fever, and yellow fever.

Without wishing to be held to a particular theory, it is contemplated that some embodiments may maintain anti-bacterial immune surveillance in an otherwise immune compromised subject. For example, a subject suffering from a viral or other immune compromising condition may normally exhibit reduced bacterial resistance, however, with administration of provided compositions may reduce or eliminate the degree of reduced bacterial resistance exhibited by the subject. In some embodiments, provided compositions are administered at regular intervals in order to maintain anti-bacterial immune surveillance. In some embodiments, In some embodiments, provided compositions are administered to a subject suffering from or susceptible to a non-bacterial immune challenge. In some embodiments, provided compositions are administered to a subject that has recently suffered from a non-bacterial immune challenge.

Cancer

In some embodiments, the subject in need thereof is suffering from a cancer including, but not limited to acute lymphoblastic leukemia (ALL); adrenocortical carcinoma; AIDS-related cancers including AIDS-related lymphoma; anal cancer; appendix cancer; astrocytomas; basal cell carcinoma; bile duct cancer; bladder cancer; bone cancer (e.g., osteosarcoma and malignant fibrous histiocytoma); brainstem glioma; brain cancer; brain tumors; breast cancer; bronchial adenomas/carcinoids; Burkitt lymphoma; carcinoid tumors (e.g., childhood and gastrointestinal tumors); carcinoma (including carcinoma of unknown primary (CUP) whose origin or developmental lineage is unknown but that possess specific molecular, cellular, and histological characteristics of epithelial cells); central nervous system lymphoma; cerebellar astrocytoma; malignant glioma; cervical cancer; childhood cancers; chronic lymphocytic leukemia; chronic myelogenous leukemia; chronic myeloproliferative disorders; colon Cancer; cutaneous T-cell lymphoma; desmoplastic small round cell tumor; endometrial cancer; ependymoma; esophageal cancer; Ewing's sarcoma in the Ewing family of tumors; extracranial germ cell tumor; extragonadal germ cell tumor; ovarian germ cell tumor; extrahepatic bile duct cancer; eye cancer; intraocular melanoma; retinoblastoma; gallbladder cancer; gastric cancer; gastrointestinal carcinoid tumor; gastrointestinal stromal tumor (GIST); gestational trophoblastic tumor; gastric carcinoid; hairy cell leukemia; head and neck cancer; heart cancer; hepatocellular (liver) cancer; Hodgkin lymphoma; hypopharyngeal cancer; hypothalamic and visual pathway glioma; intraocular Melanoma; Islet Cell Carcinoma (Endocrine Pancreas); kaposi sarcoma; soft tissue sarcoma; uterine sarcoma; kidney cancer (renal cell carcinoma); laryngeal cancer; leukemias (including acute lymphoblastic or acute lymphocytic leukemia, acute myeloid or acute myelogenous leukemia, chronic lymphocytic or chronic lymphocytic leukemia, chronic myelogenous or chronic myeloid leukemia); Lip and Oral Cavity Cancer; liposarcoma; liver cancer; lung cancer (including non-small cell and small cell); lymphomas (e.g., AIDS-related, Burkitt, cutaneous T-Cell, Hodgkin, non-Hodgkin, Primary Central Nervous System); macroglobulinemia; medulloblastoma; melanoma; Merkel Cell Carcinoma; mesothelioma (e.g., adult malignant mesothelioma, childhood mesothelioma); metastatic squamous neck cancer; mouth cancer; Multiple Endocrine Neoplasia Syndrome; Multiple Myeloma; Mycosis Fungoides; Myelodysplastic Syndromes; Myelodysplastic/Myeloproliferative Diseases; Myelogenous Leukemia; Myeloid Leukemia; (e.g., Adult Acute; nasal cavity and paranasal sinus cancer; nasopharyngeal carcinoma; neuroblastoma; oral cancer; oropharyngeal cancer; ovarian cancer; ovarian epithelial cancer (Surface epithelial-stromal tumor); ovarian germ cell tumor; ovarian low malignant potential tumor; pancreatic cancer; paranasal sinus and nasal cavity cancer; parathyroid cancer; penile cancer; pharyngeal cancer; pheochromocytoma; pineal astrocytoma; pineal germinoma; pineoblastoma and supratentorial primitive neuroectodermal tumors; pituitary adenoma; pleuropulmonary blastoma; prostate cancer; rectal cancer; renal pelvis and ureter and transitional cell cancer; rhabdomyosarcoma; Sezary syndrome; skin cancer (including melanoma and nonmelanoma); skin carcinoma; small intestine cancer; squamous cell carcinoma; stomach cancer; testicular cancer; throat cancer; thymoma and thymic carcinoma; thyroid cancer; urethral cancer; endometrial uterine cancer; vaginal cancer; vulvar cancer; and/or combinations thereof.

Autoimmune Diseases, Disorders, and/or Conditions

Autoimmune diseases, disorders and/or conditions are characterized by failure of an organism to recognize its own constituent parts as “self,” resulting in an immune response against an organism's own tissues, cells and entities. In some embodiments, the subject in need thereof is suffering from at least one autoimmune disease, disorder and/or condition. In some embodiments, an autoimmune disease, disorder and/or condition is characterized by one or more symptoms including, but not limited to autoantibodies to one or more of the subject's own tissues or bodily fluids; inflammation; swelling; heat, redness and/or pain of one or more bodily tissues; generalized malaise; weakness; joint pain; joint stiffness; loss of function of one or more joints; fever; weight loss; weight gain; dysfunction of one or more bodily (e.g. endocrine, cardiovascular, etc.) or organ systems (e.g. kidney, gastrointestinal, etc.); rashes; blisters; pigment changes of the skin; headaches; migraines; visual and/or balance disturbances or dysfunction; vertigo; and/or combinations thereof. Without wishing to be limited, autoimmune diseases, disorders and/or conditions may include one or more of: acute disseminated encephalomyelitis (ADEM); Addison's disease; alopecia universalis; ankylosing spondylitisis; antiphospholipid antibody syndrome (APS); aplastic anemia; autoimmune hemolytic anemia; autoimmune hepatitis; autoimmune inner ear disease (AIED); autoimmune lymphoproliferative syndrome (ALPS); autoimmune oophoritis; Balo disease; Behcet's disease; Bullous pemphigoid; cardiomyopathy; Chagas' disease; chronic fatigue immune dysfunction syndrome (CFIDS); chronic inflammatory demyelinating polyneuropathy; Crohn's disease; cicatrical pemphigoid; coeliac sprue-dermatitis herpetiformis; cold agglutinin disease; CREST syndrome; Degos disease; diabetes mellitus; discoid lupus; dysautonomia; endometriosis; essential mixed cryoglobulinemia; fibromyalgia-fibromyositis; Goodpasture's syndrome; Grave's disease; Guillain-Barre syndrome (GBS); Hashimoto's thyroiditis; Hidradenitis suppurativa; idiopathic and/or acute thrombocytopenic purpura; idiopathic pulmonary fibrosis; IgA neuropathy; interstitial cystitis; juvenile arthritis; Kawasaki's disease; Lichen planus; Lupus erythematosus; Lyme disease; Meniere disease; Mixed connective tissue disease (MCTD); multiple sclerosis; myasthenia gravis; neuromyotonia; opsoclonus myoclonus syndrome (OMS); optic neuritis; Ord's thyroiditis; pemphigus vulgaris; pernicious anemia; polyarthritis; polychondritis; polymyositis and dermatomyositis; primary biliary cirrhosis; psoriasis; polyarteritis nodosa; polyglandular syndromes; polymyalgia rheumatic; primary agammaglobulinemia; Raynaud phenomenon; rheumatoid arthritis; Reiter's syndrome; rheumatic fever; sarcoidosis; schizophrenia; scleroderma; Sjögren's syndrome; stiff person syndrome; Takayasu's arteritis; temporal arteritis (also known as “giant cell arteritis”); ulcerative colitis; uveitis; vasculitis; vitiligo; vulvodynia (“vulvar vestibulitis”); and Wegener's granulomatosis. One of skill in the art will recognize that the preceding list is exemplary, and not to be considered a comprehensive list of known or suspected autoimmune diseases, disorders or conditions.

Graft Versus Host Disease

In some embodiments, the subject in need thereof is suffering from graft versus host disease (GVHD). GVHD is a complication that may occur following an allogeneic or xenogenic tissue transplant (e.g. tissue implantation, graft, etc.), wherein transplanted donor immune cells (e.g., white blood cells including T cells) present in the graft (i.e. donor) tissue recognize the host (i.e., recipient) as “non-self” (i.e., antigenically “foreign”). This recognition as “non-self” results in attack of the tissues of the recipient, ultimately resulting in donor immune cells attacking and damaging and/or destroying cells of the host.

In some embodiments, the subject suffering from GVHD (host) has received one or more tissue grafts from a donor, wherein the one or more tissue grafts comprise viable and functional immune cells. In some embodiments, the one or more tissue grafts comprises bone marrow, organs or portions thereof, cells and/or combinations thereof. In some embodiments, the host is immunocompromised, wherein the host does not have cells capable of destroying or inactivating grafted donor immune cells. In some embodiments, GVHD is acute. In some embodiments, GVHD is chronic. In some embodiments, GVHD is considered acute if the subject suffering from GVHD has received a transplant from a donor within about 3 months from the appearance of symptoms. In some embodiments, GVHD is considered chronic if the subject suffering from GVHD has received a transplant from a donor greater than about 3 months from the appearance of symptoms. In some embodiments, symptoms of acute GVHD include but are not limited to nausea; vomiting; diarrhea; abdominal pain; abdominal cramps; liver dysfunction or malfunction (e.g. jaundice, etc.); rash; redness and/or itching of the skin; and/or combinations thereof. In some embodiments, symptoms of chronic GVHD include but are not limited changes in the eye (e.g. dry eyes, visual disturbances or changes, etc.); changes in the mouth (e.g. white patches, ulcerations, sensitivity to certain foods that the subject was not previously sensitive to (e.g. citrus, spice, etc.)); fatigue; weakness; muscle weakness; chronic pain; joint pain; joint stiffness; changes in the skin (e.g. rash, skin tightening, areas of thickening, raised areas, discoloration, etc.); shortness of breath, lung stiffening, eye inflammation, joint contractures, difficulty swallowing; weight loss; vaginal dryness; and/or combinations thereof.

Host Versus Graft Disease

In some embodiments, the subject in need thereof is suffering from Host-versus-graft disease (i.e., transplant rejection; graft rejection, HvGD). Host-versus-graft disease occurs when tissue transplanted from a donor (e.g., tissue transplantation, implantation, graft, etc.) into a host is rejected by the immune system of the host. This results in an attack on the donor tissue (by the host's immune system), wherein the host immune system recognizes the donor cells as different, thereby attacking and destroying them. In some embodiments, the severity and/or type of transplant rejection may vary and includes hyperacute rejection; acute rejection; chronic rejection; and/or combinations thereof.

In some embodiments, symptoms of hyperacute rejection may appear within minutes after a recipient receives a transplant, wherein the only treatment is immediate removal of the transplanted tissue. In some embodiments, hyperacute rejection may result in systemic inflammatory response syndrome (SIRS), which is a condition characterized by symptoms including, e.g. systemic inflammation, organ dysfunction, and organ failure which may include abnormal expression and/or regulation of various cytokines (SIRS is a subset of a cytokine storm).

In some embodiments, acute rejection may occur any time from the first week after transplant to three or more months after transplant, and, to some degree, occurs in all transplants unless proper immunomodulation is achieved. In some embodiments acute rejection may be treated with immunomodulatory therapy; immunosuppressive therapy; antibody-based therapy; bone marrow transplant; and/or combinations thereof.

In some embodiments, acute rejection may be a single episode or multiple episodes. In some embodiments, recurrent episodes of acute rejection may lead to chronic rejection. Chronic rejection is rejection that occurs over a time period of years and is characterized by a host's constant immune response against a transplant. The constant rejection ultimately results in cumulative damage to the transplant, including, as fibrosis (i.e. scarring), which can lead to dysfunction and failure.

A variety of methods for diagnosing acute and/or chronic rejection are well known in the art and include use of clinical data (e.g. patient signs and symptoms) as well as pathology data (e.g., tissue biopsy, imaging, etc.).

Dosing

In some embodiments, provided compositions are administered according to a dosing regimen sufficient to achieve a desired immunological reaction. For example, in some embodiments, a dosing regimen is sufficient to achieve a desired immunological reaction if its administration to a relevant patient population shows a statistically significant correlation with achievement of the desired immunological reaction (e.g., a predominantly Th1- or T_{reg} response).

In some embodiments, the desired immunological reaction is a reduction in the degree and/or prevalence of symptoms of allergy of at least about 20%, about 25%; about 30%; about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In some embodiments, a provided composition is administered according to a dosing regimen sufficient to achieve a reduction in the degree and/or prevalence of symptoms of allergy of a specified percentage of a population of patients to which the composition is administered. In some embodiments, the specified percentage of population of patients to which the composition was administered is at least about 5%, about 10%, about 15%, about 20%, about 25%, about 30%, about 31%, about 32%. about 33%. about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

To give but a few illustrative examples, in some embodiments, administration of at least one provided composition according to a dosing regimen is sufficient to achieve a reduction in the degree and/or prevalence of allergy of at least about 20% in at least about 50% of the population of patients to which the composition was administered. In some embodiments, administration of at least one provided composition according to a dosing regimen is sufficient to achieve a reduction in the degree and/or prevalence of allergy of at least about 30% in at least about 50% of the population of patients to which the composition was administered.

In some embodiments, at least one provided pharmaceutical composition is administered according to a dosing regimen sufficient to achieve a delay in the onset of symptoms of allergy. In some embodiments, at least one provided composition is administered according to a dosing regimen sufficient to prevent the onset of one or more symptoms of allergy.

In some embodiments, a provided dosing regimen comprises or consists of a single dose. In some embodiments, a provided dosing regimen comprises or consists of multiple doses, separated from one another by intervals of time that may or may not vary. In some embodiments, a provided dosing regimen comprises or consists of dosing once every 20 years, once every 10 years, once every 5 years, once every 4 years, once every 3 years, once every 2 years, once per year, twice per year, 3 times per year, 4 times per year, 5 times per year, 6 times per year, 7 times per year, 8 times per year, 9 times per year, 10 times per year, 11 times per year, once per month, twice per month, three times per month, once per week, twice per week, three times per week, 4 times per week, 5 times per week, 6 times per week, daily, twice daily, 3 times daily, 4 times daily, 5 times daily, 6 times daily, 7 times daily, 8 times daily, 9 times daily, 10 times daily, 11 times daily, 12 times daily, or hourly.

In some embodiments, a provided dosing regimen comprises or consists of an initial dose with one or more booster doses. In some embodiments, one or more booster doses are administered 1 day, 2 days, 3 days, 4 days, 5 days, 6 days, 1 week, 2 weeks, 1 month, 2 months, 6 months, 1 year, 2 years, 5 years, 10 years, or longer than 10 years after the initial dose. In some embodiments, an initial dose comprises a series of doses administered over a period of time. For example, in some embodiments, an initial dose comprises a series of 1, 2, 3, 4, 5, 6, 7, 8, 9, 10, 11, 12, 13, 14, 15, 16, 17, 18, 19, 20, 21, 22, 23, 24, or more doses administered at regular intervals, e.g., intervals that are close in time to one another, such as 5 minute intervals, 10 minute intervals, 15 minute intervals, 20 minute intervals, 25 minute intervals, 30 minute intervals, 45 minute intervals, hourly intervals, every 2 hours, etc.

In some embodiments, an initial dose and booster doses contain the same amount of provided composition. In some embodiments, an initial dose and booster doses contain different amounts of provided composition. In certain embodiments, provided compositions at dosage levels sufficient to deliver from about 0.001 mg/kg to about 100 mg/kg, from about 0.01 mg/kg to about 50 mg/kg, preferably from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day. In some embodiments, provided compositions are formulated into a unit dose. In some embodiments, a unit dosage is about 10 mg, about 25 mg, about 50 mg, about 100 mg, about 250 mg, about 500 mg, about 1 g, about 5 g, about 10 g, about 25 g, about 50 g, about 100 g, or more than about 100 g. In some embodiments, the amount of provided composition present in a particular unit dose depends on the subject to which the composition is to be administered. To give but a few examples, in some embodiments, a unit dose appropriate for a mouse is smaller than a unit dose that is appropriate for a rat, which is smaller than a unit dose that is appropriate for a dog, is smaller than a unit dose that is appropriate for a human.

In some embodiments, a provided dosing regimen comprises or consists of administration of multiple doses over the course of the subject's entire lifespan. In some embodiments, a provided dosing regimen comprises administration of multiple doses over the course of several years (e.g., 2, 3, 4, 5, 6, 7, 8, 9, 10, 15, 20, 25, 30, 35, 40, 45, 50, 60, 70, 80, 90, or 100 years). In some embodiments, a provided dosing regimen comprises or consists of multiple doses over the course of 1, 2, 3, 4, 5, 6, 7, 8, 9, 10, 11, or 12 months.

In some embodiments, when provided compositions are used in the treatment of allergy, prior to the first dose, a subject's baseline allergic response is determined by one or more of a variety of methods, including, but not limited to, (1) performing a prick skin test (PST) of one or more of the subject's 1, 2, 3, 4, 5, 6, 7, 8, 9, 10, 11, 12, 13, 14, 15, 16, 17, 18, 19, 20, or more than 20 allergens, and measuring the wheal and flare response to the PST; (2) measuring blood serum IgE levels; (3) noting the subject's own description of her typical symptoms (e.g., nature, severity, and/or duration of symptoms) upon exposure to one or more of her 1, 2, 3, 4, 5, 6, 7, 8, 9, 10, 11, 12, 13, 14, 15, 16, 17, 18, 19, 20, or more than 20 allergens; (4) exposing the subject to a certain dose of one or more of her 1, 2, 3, 4, 5, 6, 7, 8, 9, 10, 11, 12, 13, 14, 15, 16, 17, 18, 19, 20, or more than 20 allergens (e.g., if only a small or nonexistent risk of anaphylaxis); (5) measuring expression (e.g., levels, spatial distribution, temporal distribution, etc.), of one or more molecular markers, including, but not limited to, T-cell markers CD4+ and/or CD8+; (6) performing a basophil histamine release assay; and/or combinations thereof. In some embodiments, a subject's allergic response is monitored using any combination of methods, e.g. methods (1)-(6) described above, throughout the course of the treatment regimen and/or after the treatment regimen is completed, e.g., at regular intervals. In some embodiments, allergic response is monitored daily, weekly, bi-weekly, monthly, 6 times per year, 4 times per year, 3 times per year, 2 times per year, once per year, every 2 years, every 5 years, and/or every 10 years, etc.

In some embodiments, a subject is challenged with a single allergen and/or multiple allergens, e.g., a subset of the subject's allergens (e.g., allergens to which the subject is known to be allergic) and/or all of the subject's allergens (e.g., allergens to which the subject is known to be allergic). In some embodiments, allergy challenge is performed after 1 week, 2 weeks, 1 month, 2 months, 6 months, and 1 year after initiation of treatment.

In some embodiments, provided compositions may be administered via any medically acceptable route. For example, in some embodiments, a provided composition may be administered via intravenous administration; intradermal administration; transdermal administration; oral administration; subcutaneous administration; transmucosal administration; and/or combinations thereof. In some embodiments, exemplary routes of transmucosal administration include, but are not limited to buccal administration; nasal administration; bronchial administration; vaginal administration; rectal administration; sublingual administration; and/or combinations thereof.

Combination Therapy

In some embodiments, provided compositions are administered to a subject in combination with one or more other therapeutic agents or modalities, for example, useful in the treatment of one or more diseases, disorders, or conditions treated by the relevant provided pharmaceutical composition, so the subject is simultaneously exposed to both. In some embodiments, a provided composition is utilized in a pharmaceutical formulation that is separate from and distinct from the pharmaceutical formulation containing the other therapeutic agent. In some embodiments, a provided composition is admixed with the composition comprising the other therapeutic agent. In other words, in some embodiments, a provided composition is produced individually, and the provided composition is simply mixed with another composition comprising another therapeutic agent.

The particular combination of therapies (substances and/or procedures) to employ in a combination regimen will take into account compatibility of the desired substances and/or procedures and the desired therapeutic effect to be achieved. In some embodiments, provided compositions can be administered concurrently with, prior to, or subsequent to, one or more other therapeutic agents (e.g., desired known allergy therapeutics).

It will be appreciated that the therapies employed may achieve a desired effect for the same disorder (for example, a provided composition useful for treating allergy may be administered concurrently with a known allergy therapeutic that is also useful for treating allergy), or they may achieve different effects (for example, a provided composition that is useful for treating allergy may be administered concurrently with a therapeutic agent that is useful for alleviating adverse side effects, for instance, inflammation, nausea, etc.). In some embodiments, provided compositions in accordance with the disclosure are administered with a second therapeutic agent that is approved by the U.S. Food and Drug Administration (FDA).

As used herein, the terms “in combination with” and “in conjunction with” mean that the provided composition(s) can be administered concurrently with, prior to, or subsequent to, one or more other desired therapeutics. In general, each substance will be administered at a dose and/or on a time schedule determined for that agent.

For example, in some embodiments, provided compositions for the treatment of allergy may, in some embodiments, be administered in combination with, for example, one or more antihistamines (i.e., histamine antagonist), corticosteroids including glucocorticoids; epinephrine (adrenaline); theophylline (dimethylxanthine); cromolyn sodium; anti-leukotrienes; anti-cholinergics; decongestants; mast cell stabilizers; immunotherapy (progressively larger doses of a specific allergen); monoclonal anti-IgE antibodies (e.g., omalizumab); and/or combinations thereof.

Exemplary antihistamines include, but are not limited to Azelastine; Brompheniramine; Buclizine; Bromodiphenhydramine; Carbinoxamine; Cetirizine; Cyclizine; Chlorpheniramine; Chlorodiphenhydramine; Clemastine; Cyproheptadine; Desloratadine; Dexbrompheniramine; Deschlorpheniramine; Dexchlorpheniramine; Dimetindene; Diphenhydramine (Benadryl); Doxylamine; Ebastine; Embramine; Fexofenadine; Levocetirizine; Loratadine; Olopatadine (Patanol); Phenindamine (Nolahist and Thephorin); Pheniramine (Avil); Phenyltoloxamine; Promethazine; Pyrilamine; Rupatadine; Tripelennamine; Triprolidine; and/or combinations thereof.

Exemplary corticosteroids and glucocorticoids include, but are not limited to Beclometasone dipropionate and Beclomethasone (Clenil, Qvar, Beconase AQ, Alanase, Vancenase); Budesonide (Rhinocort, Rhinosol, Pulmicort, Budicort, Symbicort, Noex); Ciclesonide (Alvesco, Omnaris, Omniair); Flunisolide (Aerobid); Fluticasone (Veramyst); Fluticasone (Flonase); Mometasone and Mometasone furoate (Nasonex); Triamcinolone (Nasacort AQ); Prednisone; Methylprednisolone (Depo-Medrol); Triamcinolone (Kenalog); and/or combinations thereof.

Exemplary forms of cromolyn sodium include, but are not limited to, Rynacrom; Nasalcrom; Prevalin; Intal; Optocrom; Optrex; Gastrocrom; Intercron; and/or combinations thereof.

Exemplary anti-leukotrienes and leukotriene inhibitors (or modifiers) include, but are not limited to Montelukast (Singulair, Montelo-10, and Monteflo); Zafirlukast (Accolate, Accoleit, Vanticon); Pranlukast; Zileuton (Zyflo, Zyflo CR); and/or combinations thereof.

Exemplary anti-cholinergics include, but are not limited to, Ipratropium bromide (Atrovent®, Apovent, Ipraxa, Aervoent); Combivent (Ipratropium bromide and Albuterol); Benztropine (Cogentin); Oxitropium (Oxivent); Tiotropium (Spiriva); Glycopyrrolate (Robinul); Oxybutinin (Ditropan, Driptane, Lyrinel XL); Tolterodine (Detrol, Detrusitol); Chlorphenamine (Chlor-Trimeton); Diphenhydramine (Benadryl, Sominex, Advil PM, etc.); Dimenhydrinate (Dramamine); Bupropion (Zyban, Wellbuterin); Hexamethonium; Tubocurarine; Dextromethorphan; Mecamylamine; Doxacurium; and/or combinations thereof.

Exemplary decongestants include, but are not limited to, Ephedrine; Levo-methamphetamine; Naphazoline; Oxymetazoline; Phenylephrine; Phenylpropanolamine; Propylhexedrine; Synephrine; Tetrahydrozoline; and/or combinations thereof.

Exemplary mast cell stabilizers include, but are not limited to, Cromoglicic acid; Ketotifen and Ketotifen fumarate (Zaditor, Zaditen, Alaway, Zyrtec Itchy-Eye Drops, Claritin Eye); Methyl xanthines; and/or combinations thereof.

In some embodiments, exemplary known allergy therapeutics that can be administered in combination with provided compositions in accordance with the disclosure include, but are not limited to, any of the therapeutics described in U.S. Pat. Nos. 5,558,869, 5,973,121, 6,835,824, 6,486,311, and/or 7,485,708, and/or in US Patent Publication Numbers 2003/0035810, 2003/0202980, 2004/0208894, 2004/0234548, 2007/0213507, 2010/0166802, and/or 2011/0027298, all of which are incorporated herein by reference.

As an additional example, in some embodiments, provided pharmaceutical compositions for the treatment of infectious disease may be administered in combination with, for example, an antibiotic such as an antibacterial agent, an antiviral agent, and/or an antifungal agent. In some embodiments, provided pharmaceutical compositions may be administered in combination with a vaccine. In some embodiments, provided pharmaceutical compositions may be administered in the absence of a vaccine.

Exemplary antibacterial agents include, but are not limited to sulfaniliamide; folic acid analogs; beta-lactams such as penicillins, cephalosporins, and carbapenems; aminoglycosides such as streptomycin, kanamycin, neomycin, and gentamycin; tetracyclines such as chlortetracycline, oxytetracycline, and doxycycline; macrolides; lincosamides; streptogramins; fluoroquinolones, rifampin, mupirocin, cycloserine, aminocyclitols, glycopeptides, oxazolidinones, and derivatives/analogs and/or combinations thereof.

Exemplary antiviral agents include, but are not limited to Abacavir, Aciclovir, Acyclovir, Adefovir, Amantadine, Amprenavir, Ampligen, Arbidol, Atazanavir, Atripla, Boceprevirertet, Cidofovir, Combivir, Darunavir, Delavirdine, Didanosine, Docosanol, Edoxudine, Efavirenz, Emtricitabine, Enfuvirtide, Entecavir, Entry inhibitors, Famciclovir, Fomivirsen, Fosamprenavir, Foscarnet, Fosfonet, Imunovir, Idoxuridine, Imiquimod, Indinavir, Inosine, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir, Peginterferon alfa-2a, Penciclovir, Peramivir, Pleconaril, Podophyllotoxin, Raltegravir, Reverse transcriptase inhibitors, Ribavirin, Rimantadine, Ritonavir, Pyramidine, Saquinavir, Stavudine, Tea tree oil, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, and derivatives/analogs and/or combinations thereof.

Exemplary antifungal agents include, but are not limited to polyene agents such as amphotericin, candicidin, filipin, hamycin, natamycin, nystatin, and rimocidin; imidazole, triazole and thiazole agents such as bifonazole, butoconazole, clotrimazole, econazole, fenticonazole, isoconazole, ketoconazole, miconazole, omoconazole, oxiconazole, sertaconazole, sulconazole, tioconazole, alboconazole, fluconazole, isavuconazole, posaconazole, ravuconazole, terconazole, voriconazole, and abafungin; allylamines such as amorolfin, butenafine, naftafine, and terbinafine; and echinocandins such as anidulafungin, caspofungin, and micafungin and derivatives/analogs and/or combinations thereof.

As an additional example, in some embodiments, provided compositions for the treatment of cancer may be administered in combination with, for example, alkylating agents, antimetabolite agents, and/or other anticancer medications.

Exemplary alkylating agents include, but are not limited to polyfunctional alkylating agents such as cyclophosphamide (Cytoxan), mechlorethamine, melphan (Alkeran), chlorambucil (Leukeran), thiopeta (Thioplex), and busulfan (Myleran); procarbazine, dacarbazine, altretamine, cisplatin, and derivatives/analogs and/or combinations thereof.

Exemplary antimetabolite agents include, but are not limited to methotrexate; purine antagonists such as mercaptopurine (6-MP), thioguanine (6-TG), fludarabine phosphate, cladribine, and pentostatin; pyrimidine antagonists such as fluorouracil, cytarabine, and azacitidine; plant alkaloids such as vinblastine (Velban), vincristine (Oncovin), etoposide (VP-16), teniposide (Vimon), topotecan (Hycamtin), irinotecan (Camptosar), paclitaxel (Taxol), and docetaxel (Taxotere) and derivatives/analogs and/or combinations thereof.

Exemplary other anticancer agents include, but are not limited to amsacrine; hydroxyurea (Hydrea); asparaginase (El-spar); mitoxantrone (Novantrone); mitotane; retinoic acid, bone marrow growth factors, amifostine, and derivatives/analogs and/or combinations thereof.

Kits

The present disclosure provides kits comprising vaccine and/or therapeutic compositions including provided compositions. In some embodiments, a kit may comprise (i) at least one provided composition; and (ii) at least one pharmaceutically acceptable excipient; and, optionally, (iii) instructions for use.

In some embodiments, kits include multiple (e.g., 1, 2, 3, 4, 5, 6, 7, 8, 9, 10, 11, 12, 13, 14, 15, 16, 17, 18, 19, 20, or more) doses of provided compositions. In some embodiments, kits include multiple (e.g., 1, 2, 3, 4, 5, 6, 7, 8, 9, 10, 11, 12, 13, 14, 15, 16, 17, 18, 19, 20, or more) populations of provided compositions having different functional elements (e.g., microbial mimic entities). In some embodiments, multiple populations of provided compositions are packaged separately from one another in provided kits. In some embodiments, provided kits may include provided compositions and one or more other therapeutic agents intended for administration with the provided compositions.

In some embodiments, the present disclosure provides pharmaceutical packs or kits including provided compositions to be used in treatment methods according to the present disclosure. In some embodiments, pharmaceutical packs or kits include preparations or pharmaceutical compositions containing provided compositions in one or more containers filled with optionally one or more additional ingredients of pharmaceutical compositions in accordance with the disclosure. In some embodiments, the pharmaceutical pack or kit includes an additional approved therapeutic agent for use in combination therapies, as described herein. Optionally associated with such container(s) can be a notice in the form prescribed by a governmental agency regulating the manufacture, use or sale of pharmaceutical products, which notice reflects approval by the agency of manufacture, use, or sale for human administration.

In some embodiments, kits are provided that include provided compositions and instructions for use. Pharmaceutical doses or instructions therefor may be provided in a kit for administration to an individual suffering from and/or susceptible to allergy.

Administration Portion or Subpopulation of Subject's Cells

According to various embodiments, provided compositions may be administered to only a portion or subpopulation of a subject's cells. By way of non-limiting example, provided compositions may be administered to a population of a subject's immune cells, for example, antigen presenting cells. In some embodiments, such administration may be ex vivo (e.g., in vitro, such as in a clinical or laboratory setting) and the population of the subject's cells may then be reintroduced to the subject.

Methods of Manufacturing

In some embodiments, the present disclosure also provides for methods of manufacturing provided compositions (e.g., compositions and/or devices containing one or more crude antigens and/or adjuvant agents as described herein). In some embodiments, provided methods include associating at least one activating antigen with at least one adjuvant agent. In some embodiments, the at least one activating agent is a Th2-associated agent. In some embodiments, the at least one activating agent is a Th1-associated agent. In some embodiments, the present disclosure provides methods of manufacturing compositions including providing at least one antigen (e.g., a Th2-associated antigen) and associating the at least one antigen with at least one adjuvant agent (e.g., a crude adjuvant agent). In some embodiments, provided methods allow for associating at least one adjuvant and at least one antigen such that they are substantially co-localized in an article. In some embodiments, the method provides for inclusion of at least one article in which the at least one antigen and the at least one adjuvant agent are substantially co-localized in one or more compartments. In some embodiments, provided methods include the physical separation of at least one antigen from at least one adjuvant agent, for example, with each being in a separate compartment of one or more articles.

Identification and/or Characterization

In some embodiments, the present disclosure provides technologies for identifying and/or characterizing useful compositions and/or methods as described herein. For example, in some embodiments, compositions of interest may be characterized in that they show a particular attribute (e.g. expression of a particular cytokine, cytokine profile, reduction of symptoms, etc.) when assayed, for example, in an in vitro cytokine release assay, a clinical trial wherein a composition of interest is administered to a subject in need thereof, etc. In some embodiments, characterization of disclosed compositions includes assessment of character and/or quality of a composition. In some embodiments, provided technologies may be utilized to identify a composition (or component) of interest. For example, a composition may be analyzed to determine if the antigen of interest is present and/or if it is released in an expected manner.

In some embodiments, a composition may be analyzed to determine if the composition (or a component) results in release of a particular cytokine and/or subset of cytokines. In some embodiments, provided technologies are assays to determine if a composition results in a certain type (e.g. Th1, Th2, etc.) of immune response. In some embodiments, provided technologies can determine if one or more elements of an immune response are present after contact with one or more disclosed compositions, articles, etc. In some embodiments, effects of disclosed compositions are determined by comparison to one or more relevant controls (e.g presence of a reference composition, absence of a test composition, etc.). In some embodiments, effects are determined by comparison of one or more characteristics of a subject in need to whom a disclosed composition has been administered (e.g. serum/blood cytokine profile, allergic symptoms, etc.). In some embodiments, effects are determined by comparing subjects in need to whom disclosed compositions were administered versus subjects to whom reference or control compositions were administered. In some embodiments, one or more disclosed compositions is characterized by ability to immunomodulate a subject in need thereof, treated with one or more of the disclosed compositions. In some embodiments, the disclosed compositions are characterized as successful if they achieve immunomodulation, further characterized by improvement of one or more symptoms of the disease or condition that the subject is suffering from.

### EQUIVALENTS AND SCOPE

Those skilled in the art will recognize, or be able to ascertain using no more than routine experimentation, many equivalents to the specific embodiments of the disclosure described herein.

We claim:

1. A composition comprising:
at least one antigen substantially co-localized with at least one adjuvant agent in a format selected from patch, capsule, tablet, gel, matrix, paste, reservoir, adhesive, liquid, suspension, lyophilized solid, liquid contained within one or more lumenal units, and combinations thereof, wherein one or both of the antigen and the adjuvant agent is a crude preparation, and wherein the composition is characterized in that administration to a subject in need thereof results in an immune response in the subject to the antigen.

2. The composition of claim 1, wherein the antigen is a Th2 associated antigen and the immune response is not a predominantly Th2 response.

3. The composition of claim 1, wherein the antigen is a Th1 associated antigen and the immune response is not a predominantly Th1 response.

4. The composition of claim 1, designed and constructed to deliver both the antigen and the adjuvant to a population of the subject's cells.

5. The composition of claim 4, wherein the population of the subject's cells is or comprises immune cells.

6. The composition of claim 5, wherein the immune cells are or comprise antigen presenting cells.

7. The composition of claim 2, wherein the immune response is a predominantly Th1 response.

8. The composition of claim 3, wherein the immune response is a predominantly Th2 response.

9. The composition of claim 7 or 8, wherein the immune response includes a Treg response.

10. The composition of claim 7, wherein a Th1 response is or comprises decreased activity of one or more of IL-4, IL-5, IL-6, IL-9, IL-10, IL-13, IgE, and histamines relative to administration of a Th2-associated antigen to the subject without the at least one adjuvant agent.

11. The composition of claim 7, wherein a Th1 response is or comprises increased activity of one or more of IFN-γ, IL-2, TNF-β, IL-12, and IgG2a relative to administration of a Th2-associated antigen to the subject without the at least one adjuvant agent.

12. The composition of claim 8, wherein a Th2 response is or comprises increased activity of one or more of IL-4, IL-5, IL-6, IL-9, IL-10, IL-13, IgE, and histamines relative to administration of a Th1-associated antigen to the subject without the at least one adjuvant agent.

13. The composition of claim 8, wherein a Th2 response is or comprises decreased activity of one or more of IFN-γ, IL-2, TNF-β, IL-12, and IgG2a relative to administration of a Th1-associated antigen to the subject without the at least one adjuvant agent.

14. The composition of claim 2 or claim 3, wherein the associated antigen is or comprises an allergic antigen.

15. The composition of claim 2 or claim 3, wherein the associated antigen is or comprises an anaphylactic antigen.

16. The composition of claim 2 or claim 3, wherein the associated antigen is or comprises an infectious antigen.

17. The composition of claim 2 or claim 3, wherein the associated antigen is or comprises an autoantigen.

18. The composition of claim 2 or claim 3, wherein the associated antigen is or comprises a disease-associated antigen.

19. The composition of any one of the preceding claims, further comprising at least one pharmaceutically acceptable carrier.

20. The composition of any one of claims 1-19, wherein the co-localization comprises covalent association.

21. The composition of any one of the preceding claims, wherein at least one adjuvant agent is a crude adjuvant agent, and the at least one crude adjuvant agent is or comprises hydrophilic cellular components.

22. The composition of any one of the preceding claims, wherein at least one adjuvant agent is a crude adjuvant agent, and the at least one crude adjuvant agent is or comprises hydrophobic cellular components.

23. The composition of any one of the preceding claims, wherein at least one adjuvant agent is a crude adjuvant agent, and at least one crude adjuvant agent comprises at least one hydrophobic cellular component and at least one hydrophilic cellular component.

24. The composition of any one of claims 21-23, wherein the at least one crude adjuvant agent is or comprises one more cellular membranes and/or cellular membrane components.

25. The composition of claim 24, wherein the cellular membrane is or comprises at least one of a microbial cellular membrane.

26. The composition of claim 25, wherein the microbial cellular membrane is or comprises a bacterial cellular membrane.

27. The composition of claim 26, wherein the bacterial cellular membrane is or comprises an Escherichia coli cellular membrane.

28. The composition of any one of claims 21-27, wherein the cellular membrane components do not include transitory vesicles.

29. The composition of any one of claims 21-27, wherein the cellular membrane components do not include outer membrane vesicles.

30. The composition of any one of the preceding claims, further comprising at least one article within which at least one Th2-associated antigen or at least one Th1-associated antigen and at least one adjuvant agent are retained.

31. The composition of claim 30, wherein the article comprises one or more compartments.

32. The composition of claim 30 or 31, wherein the article is selected from a group consisting of: patch, capsule, gel, matrix, paste, reservoir, adhesive, liquid, liquid contained within one or more lumenal units, and combinations thereof.

33. The composition of claim 30 or 31, wherein the at least one Th2-associated antigen or at least one Th1-associated antigen and the at least one adjuvant agent are in the same compartment.

34. The composition of claim 30 or 31, wherein the at least one Th2-associated antigen or at least one Th1-associated antigen and the at least one adjuvant agent are in different compartments.

35. The composition of claim 32, wherein the gel is a hydrogel.

36. A method comprising
administering the composition of any one of the preceding claims to a subject in need thereof.

37. The method of claim 36, wherein the subject is suffering from a condition that results in a host immune response.

38. The method of claim 36 wherein the subject is suffering from a disease or infection.

39. The method of claim 38, wherein the subject is suffering from cancer.

40. The method of claim 36, wherein the subject is suffering or has suffered from or is susceptible to at least one allergic response to at least one antigen.

41. The method of any one of claims 36-40, wherein the composition is administered via one or more routes selected from the group consisting of bronchial instillation, and/or inhalation; buccal, enteral, interdermal, intra-arterial (IA), intradermal, intragastric (IG), intramedullary, intramuscular (IM), intranasal, intraperitoneal (IP), intrathecal, intratracheal instillation (by), intravenous (IV), intraventricular, mucosal, nasal spray, and/or aerosol, rectal (PR), subcutaneous (SQ), sublingual; topical and/or transdermal, transdermal, vaginal, vitreal, and/or through a portal vein catheter; and/or combinations thereof.

42. The method of any of claim 41, wherein the administration is topical and/or transdermal.

43. The method of any one of claims 36-42, wherein administering results in immune tolerance to the antigen.

44. The method of claim 43, wherein the tolerance is characterized by one or more of: an increased Th1 response and/or a lack of eliciting a Th2 response, wherein the Th2 response causes one or more of skin reactions wherein the skin reactions comprise one or more of hives, flushed skin, pale skin, itchiness of skin, swelling of skin; increase or feeling of increase in body temperature; tightness or feeling of tightness of the throat; constriction of airways; swelling of the tongue; swelling of the throat; difficulty breathing; weak and/or rapid pulse, nausea; vomiting; diarrhea; dizziness; fainting; and sudden or sharp decrease in blood pressure.

45. The method of any one of claims 36-44, wherein administering results in immune desensitization to the antigen.

46. The method of claim 45, wherein the desensitization is characterized by a decrease in Th2 response.

47. The method of claim 45, wherein the desensitization is characterized by an increase in Th1 response.

48. A method comprising:
associating at least one Th1-associated antigen and/or at least one Th2-associated antigen with at least one adjuvant agent.

49. A kit comprising at least one Th2-associated antigen, at least one adjuvant agent and at least one article and/or at least one pharmaceutically acceptable excipient.

50. A kit comprising at least one Th1-associated antigen, at least one adjuvant agent and at least one article and/or at least one pharmaceutically acceptable excipient.
